(19) United States
(12) Patent Application Publication
(10) Pub. No.: US 2003/0039628 A1
(43) Pub. Date: Feb. 27, 2003

(54) Activation and protection of T-cells (CD4+ and CD8+) using an H2 receptor agonist and other T-cell activating agents

(76) Inventors: Kristoffer Hellstrand, Goteborg (SE); Svante Hermodsson, Molndal (SE); Kurt R. Gehlsen, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/265,521

(22) Filed: Oct. 3, 2002

Related U.S. Application Data
(63) Continuation of application No. 09/139,281, filed on Aug. 24, 1998, now abandoned.

(51) Int. Cl. A61K 38/21; A61K 39/00; A61K 38/20

(52) U.S. Cl. 424/85.2; 424/85.5; 424/85.6; 424/85.7; 424/185.1; 424/236.1

(57) ABSTRACT

The present invention relates to a method for facilitating activation of T-cells in a patient, comprising: identifying a patient in need of enhanced T-cell activity, administering an effective amount of a T-cell activating composition to the patient, and administering an effective amount of a compound that inhibits the production or release of intercellular reactive oxygen metabolites (ROM) to the patient. The present invention further relates to the use of H₂-receptor agonists to augment the effectiveness of vaccines.

### RELATED APPLICATIONS

This is a continuation application of U.S. patent application Ser. No. 09/139,281, filed on Aug. 24, 1998, which is hereby incorporated by reference in its entirety.

### FIELD OF THE INVENTION

The present invention relates to methods of treating cancer or viral diseases in which histamine or an H₂-receptor agonist is administered alone or in conjunction with additional agents. The administration of these various agents results in the activation and protection of T-cells from the deleterious and inhibitory effects of monocytes/macrophages (MO), as well as a stimulation of the anti-cancer and anti-viral properties of T-cells. In addition, antigen presenting cells may become more effective at antigen presentation to Tcells as a direct effect of histamine or an H₂R agonist. The addition of other agents that are T-cell activation compounds which stimulate the cytotoxic activity of cytotoxic T-cells (CTLs), and other T-cell activities, preferably in a synergistic fashion with a H₂-receptor agonist are also contemplated. Representatives of such immunological stimulatory compounds include cytokines, peptides, flavonoids, vaccines, and vaccine adjuvants. Additional classes of agents usable with the methods of the present invention encompass chemotherapeutic and/or antiviral agents. The present invention also contemplates the use of reactive oxygen metabolite scavengers in conjunction with the above mentioned compounds.

### BACKGROUND OF THE INVENTION

The immune system has evolved complex mechanisms for recognizing and destroying foreign cells or organisms present in the body of the host. Harnessing the body's immune mechanisms is an attractive approach to achieving effective treatment of malignancies and viral infections.

The immune system has two types of responses to foreign bodies based on the components which mediate the response: a humoral response and a cell-mediated response. The humoral response is mediated by antibodies while the cell-mediated response involves cells classified as lymphocytes. Recent anticancer and antiviral strategies have focused on utilizing the cell-mediated host immune system as a means of anticancer or antiviral treatment or therapy. A brief review of the immune system will assist in placing the present invention in context.

Generation of an Immune Response

The immune system functions in three phases to protect the host from foreign bodies: the cognitive phase, the activation phase, and the effector phase. In the cognitive phase, the immune system recognizes and signals the presence of a foreign antigen or invader in the body. The foreign antigen can be, for example, a cell surface marker from a neoplastic cell or a viral protein. Once the system is aware of an invading body, the cells of the immune system proliferate and differentiate in response to the invader-triggered signals. The last stage is the effector stage in which the effector cells of the immune system respond to and neutralize the detected invader.

A wide array of effector cells implement an immune response to an invader. One type of effector cell, the B cell, generates antibodies targeted against foreign antigens encountered by the host. In combination with the complement system, antibodies direct the destruction of cells or organisms bearing the targeted antigen.

Another type of effector cell is the natural killer cell (NK cell), a type of lymphocyte having the capacity to spontaneously recognize and destroy a variety of virus infected cells as well as malignant cell types. The method used by NK cells to recognize target cells is poorly understood.

Another type of effector cell, the T-cell, is divided into three subcategories, each playing a different role in the immune response. Helper T-cells secrete cytokines which stimulate the proliferation of other cells necessary for mounting an effective immune response, while suppressor T-cells down regulate the immune response. A third category of T-cell, the cytotoxic T-cell (CTL), is capable of directly lysing a targeted cell presenting a foreign antigen on its surface.

The Major Histocompatability Complex and T Cell Target Recognition

T-cells are antigen specific immune cells, that function in response to specific antigen signals. B lymphocytes and the antibodies they produce are also antigen specific entities. However, unlike B lymphocytes, T-cells do not respond to antigens in a free or soluble form. For a T-cell to respond to an antigen, it requires the antigen to be bound to a presenting complex known as the major histocompatibility complex (MHC).

MHC complex proteins provide the means by which T-cells differentiate native or “self” cells from foreign cells. There are two types of MHC, class I MHC and class II MHC. T Helper cells (CD4⁺) predominately interact with class II MHC proteins while cytolytic T-cells (CD8⁺) predominately interact with class I MHC proteins. Both MHC complexes are transmembrane proteins with a majority of their structure on the external surface of the cell. Additionally, both classes of MHC have a peptide binding cleft on their external portions. It is in this cleft that small fragments of proteins, native or foreign, are bound and presented to the extracellular environment.

Cells called antigen presenting cells (APCs) display antigens to T-cells using the MHC complexes. For T-cells to recognize an antigen, it must be presented on the MHC complex for recognition. This requirement is called MHC restriction and it is the mechanism by which T-cells differentiate “self” from “non-self” cells. If an antigen is not displayed by a recognizable MHC complex, the T-cell will not recognize and act on the antigen signal.

T-cells specific for the peptide bound to a recognizable MHC complex bind to these MHC-peptide complexes and proceed to the next stage of the immune response.

Cytokines Involved In Mediating the Immune Response

The interplay between the various effector cells listed above is influenced by the activities of a wide variety of chemical factors which serve to enhance or reduce the immune response as needed. Such chemical modulators may be produced by the effector cells themselves and may influence the activity of immune cells of the same or different type as the factor producing cell.

One category of chemical mediators of the immune response is cytokines, molecules which stimulate a proliferative response in the cellular components of the immune system.

Interleukin-2 (IL-2) is a cytokine synthesized by T-cells which was first identified in conjunction with its role in the expansion of T-cells in response to an antigen (Smith, K. A. Science 240:1169 (1988)). It is well known that IL-2 secretion is necessary for the full development of cytotoxic effector T-cells (CTLs), which play an important role in the host defense against viruses. Several studies have also demonstrated that IL-2 has antitumor effects that make it an attractive agent for treating malignancies (see e.g. Lotze, M. T. et al, in “Interleukin 2”, ed. K. A. Smith, Academic Press, Inc., San Diego, Calif., p237 (1988); Rosenberg, S., Ann. Surgery 208:121 (1988)). In fact, IL-2 has been utilized to treat subjects suffering from malignant melanoma, renal cell carcinoma, and acute myelogenous leukemia. (Rosenberg, S. A., et al., N. Eng. J. Med. 316:889-897 (1987); Dutcher, J. P., et al., J. Clin. Oncol 7:477-485 (1989); Foa, R., et al., Br. J. Haematol. 77:491-496 (1991)).

Another cytokine with promise as an anticancer and antiviral agent is interferon-α. Interferon-α (IFN-α) is an IFN type I cytokine, has been employed to treat leukemia, myeloma, and renal cell carcinomas. IFN type I cytokines have been shown to increases class I MHC molecule expression. Because most cytolytic T-cells (CTLs) recognize foreign antigens bound to class I MHC molecules, type I IFNs may boost the effector phase of cell-mediated immune responses by enhancing the efficiency of CTL-mediated killing. At the same time, type I IFN may inhibit the cognitive phase of immune responses, by preventing the activation of class II MHC-restricted helper T-cells. IL-12, IL-15, and various flavonoids can also increase the T-cell response.

In vivo Results of Histamine Agonist Treatments

Histamine is a biogenic amine, i.e. an amino acid that possesses biological activity mediated by pharmacological receptors after decarboxylation. The role of histamine in immediate type hypersensitivity is well established. (Plaut, M. and Lichtenstein, L. M. 1982 Histamine and immune responses. In Pharmacology of Histamine Receptors, Ganellin, C. R. and M. E. Parsons eds. John Wright & Sons, Bristol pp. 392-435.)

Examinations of whether a H₂-receptor agonists or antagonists can be applied to the treatment of cancer have yielded contradictory results. Some reports suggest that administration of histamine alone suppressed tumor growth in hosts having a malignancy. (Burtin, Cancer Lett. 12:195 (1981)). On the other hand, histamine has been reported to accelerate tumor growth in rodents. (Nordlund, J. J., et al., J. Invest. Dermatol 81:28 (1983)).

Similarly, contradictory results were obtained when the effects of histamine-receptor antagonists were evaluated. Some studies report that histamine-receptor antagonists suppress tumor development in rodents and humans. (Osband, M. E., et al., Lancet 1 (8221):636 (1981)). Other studies report that such treatment enhances tumor growth and may even induce tumors. (Bama, B. P., et al., Oncology 40:43 (1983)).

Synergistic Effects of a H₂-Receptor Agonist and IL-2

Despite the conflicting results when histamine is administered alone, recent reports clearly reveal that histamine acts synergistically with cytokines to augment the cytotoxicity of NK cells. For example, studies using histamine analogues suggest that histamine's synergistic effects are exerted through the H₂-receptors expressed on the cell surface of monocytes. (Hellstrand, K., et al., J. Immunol. 137:656 (1986)).

Histamine's synergistic effect when combined with cytokines appears to result from the suppression of a down regulation of cytotoxicity mediated by other cell types present along with the cytotoxic cells. In vitro studies with NK cells alone confirm that cytotoxicity is stimulated when IL-2 is administered. However, in the presence of monocytes, the IL-2 induced enhancement of cytotoxicity of NK cells is suppressed. (See, U.S. Pat. No. 5,348,739, which is incorporated herein by reference).

In the absence of monocytes, histamine had no effect or weakly suppressed NK mediated cytotoxicity. (Hellstrand, K., et al., J. Immunol. 137:656 (1986); Hellstrand, K. and Hermodsson, S., Int. Arch. Allergy Appl. Immunol. 92:379-389 (1990)). Yet, NK cells exposed to histamine and IL-2 in the presence of monocytes exhibit elevated levels of cytotoxicity relative to that obtained when NK cells are exposed only to IL-2 in the presence of monocytes. Id. Thus, the synergistic enhancement of NK cell cytotoxicity by combined histamine and interleukin-2 treatment results not from the direct action of histamine on NK cells but rather from suppression of an inhibitory signal generated by monocytes.

Without being limited to a particular mechanism, it is believed that the inhibitory effects of monocytes on NK-cell cytotoxicity result from the generation of reactive oxygen metabolites such as H₂O₂ by monocytes. Hydrogen peroxide may be generated within the cell. Alternatively, H₂O₂ may be catalyzed by enzymes located on the surface of MO cells. Both sources of H₂O₂ are thought to contribute to intercellular H₂O₂ concentrations.

Granulocyte have also been shown to suppress IL-2 induced NK-cell cytotoxicity in vitro. It appears that the H₂-receptor is involved in transducing histamine's synergistic effects on overcoming granulocyte mediated suppression. For example, the effect of histamine on granulocyte mediated suppression of antibody dependent cytotoxicity of NK cells was blocked by the H₂-receptor antagonist ranitidine and mimicked by the H₂-receptor agonist dimaprit. In contrast to the complete or nearly complete abrogation of monocyte mediated NK cell suppression by histamine and IL-2, such treatment only partially removed granulocyte mediated NK cell suppression. (U.S. Pat. No. 5,348,739; Hellstrand, K., et al., Histaminergic regulation of antibody dependent cellular cytotoxicity of granulocytes, monocytes and natural killer cells., J. Leukoc. Biol 55:392-397 (1994)).

As suggested by the experiments above, therapies employing histamine and cytokines are effective anticancer and antiviral strategies. U.S. Pat. No. 5,348,739 discloses that mice given histamine and IL-2 prior to inoculation with melanoma cell lines were protected against the development of lung metastatic foci. It has also been shown that a single dose of histamine could prolong survival time in animals inoculated intravenously with herpes simplex virus (HSV), and a synergistic effect on the survival time of animals treated with a combination of histamine and IL-2 was observed (Hellstrand, K., et al., Role of histamine in natural killer cell-dependent protection against herpes simplex virus type 2 infection in mice., Clin. Diagn. Lab. Immunol. 2:277-280 (1995)).

The above results demonstrate that strategies employing a combination of histamine and IL-2 are an effective means of treating malignancies and viral infection.

Presently the therapeutic potential of several immune cell stimulating compounds that show promise as efficacious anticancer and antiviral agents is diminished due to negatively regulating systems of the immune system. Accordingly, there is a need for methods which maximize the therapeutic potential of immune cell stimulating compounds.

### SUMMARY OF THE INVENTION

The present invention relates to a method for facilitating activation of T-cells in a patient, comprising: identifying a patient in need of enhanced T-cell activity, administering an effective amount of a T-cell activating composition to the patient, and administering an effective amount of a compound that inhibits the production or release of intercellular reactive oxygen metabolites (ROM) to the patient.

The present invention further comprises a vaccine adjuvant, a vaccine, a peptide, a cytokine or a flavonoid. Vaccine adjuvants for use with the present invention may be selected from the group consisting of bacillus Calmette-Guerin (BCG), pertussis toxin (PT), cholera toxin (CT), E. coli heat-labile toxin (LT), mycobacterial 71-kDa cell wall associated protein, microemulsion MF59, microparticles of poly(lactide-co-glycolides)(PLG), and immune stimulating complexes (ISCOMS). Vaccines for use with the present invention may be selected from the group consisting of influenza vaccines, human immunodeficiency virus vaccines, Salmonella enteritidis vaccines, hepatitis B vaccines, Boretella bronchiseptica vaccines, tuberculosis vaccines, allogeneic cancer vaccines, and autologous cancer vaccines.

The present invention contemplates the use of a variety of cytokines and flavonoids. The cytokines may be selected from IL-1, IL-2, IL-12, IL-15, IFN-α, IFN-β, or IFN-γ. Flavonoids may be selected from the group consisting of flavone acetic acids and xanthenone-4-acetic acids. These compounds may be administered in a daily dose to an adult human of between 1000 and 600,000 U/kg.

The present invention further contemplates the use of compounds effective to inhibit the production or release of intercellular hydrogen peroxide selected from the group consisting of histamine, serotonin, dimaprit, clonidine, tolazoline, impromadine, 4-methylhistamine, betazole, and a histamine congener. These compounds may be administered to an adult human at between 0.05 and 50 mg per dose. These compounds may also be administered at between 1 and 500 μg/kg of patient weight per dose.

The present invention contemplates administration of the T-cell activating compound and the hydrogen peroxide scavenger administered within 1 hour thereof. Alternatively, the administration of the T-cell activating compound and the hydrogen peroxide scavenger is administered within 24 hours thereof.

The methods of the present invention further contemplate administering an effective amount of a scavenger of intercellular hydrogen peroxide. The scavenger may be selected from the group consisting of catalase, glutathione peroxidase, and ascorbate peroxidase. The hydrogen peroxide scavenger may be administered to an adult human in a dose of from about 0.05 to about 50 mg/day and the compounds maybe administered together or separately.

In addition to the compounds discussed above, the present invention contemplates the administration of a variety of chemotherapeutic agents. When the chemotherapeutic agent is an anticancer agent, the agent may be selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, epirubicine, bleomycin, nitomycin, cisplatin, carboplatin, procarbazine, amacrine, mitoxantron, tamoxifen, nilutamid, and aminoglutemide. Conventional dosages of these agents can be used.

When the chemotherapeutic agent administered is an antiviral agent, it may be selected from the group consisting of idoxuridine, trifluorothymidine, adenine arabinoside, acycloguanosine, bromovinyldeoxyuridine, ribavirin, trisodium phosphophonoformate, amantadine, rimantadine, (S)-9-(2,3-Dihydroxypropyl)-adenine, 4′,6-dichloroflavan, AZT, 3′(azido-3′-deoxythymidine), ganciclovir, didanosine (2′,3′-dideoxyinosine or ddl), zalcitabine (2′,3′-dideoxycytidine or ddC), dideoxyadenosine (ddA), nevirapine, inhibitors of the HIV protease, and other viral protease inhibitors. Conventional dosages of these agents can be used.

The methods of the present invention further contemplate the steps of administering a T-cell activating composition, a compound that inhibits the production or release of intercellular hydrogen peroxide and a chemotherapeutic agent, concomitantly.

### BRIEF DESCRIPTION OF THE DRAWINGS

FIG. 1A graphically depicts the percent of activation of CD3⁺ lymphocytes in the presence and absence of monocytes in response to IL-2 or IFN-α alone or with the H2-receptor agonist, histamine. Lymphocytes alone (lymph; open bars) or lymphocytes and monocytes (lymph+mono; filled bars) were exposed to a culture media as a control (med), IL-2 (100 U/ml), IFN-α (100 U/ml; IFN) and/or histamine (50 :M; h). Activation of CD3⁺ lymphocytes was determined by detection of CD69 expression as measured in a FACScan Flow Cytometer (Becton Dickinson, Stockholm, Sweden) using gates comprising all viable lymphocytes. The bars indicate the appearance of the CD69 cell surface marker in response to treatment, expressed as the mean of the percentage of CD69⁺presenting cells over the total CD3⁺presenting cell population±s.e.m. from up to eleven donors. Open stars (☆) refer to statistical comparisons (Mann-Whitney U-test) between cells incubated with and without MO. Filled stars (*) refer to comparisons between cells incubated with and without histamine. * or * p<0.05 (CD8⁺cells: medium with vs. without MO; CD4⁺cells: histamine with vs. without MO; CD4⁺ and CD8 cells: IL-2 with vs. without MO; CD3,⁺ cells: medium with MO vs. histamine with MO; CD4⁺ and CD8⁺ cells: IL-2 with MO vs. h+IL-2 with MO; CD4⁺ cells: IFN with MO vs. h+IFN with MO). ** or ** p<0.01 (CD3,⁺ cells: medium with vs. without MO; CD3,⁺ and CD56⁺ cells: IL-2 with vs. without MO; CD56⁺ cells: IL-2 with MO vs. h+IL-2 with MO; CD3,⁺ and CD56⁺ cells:IFN with MO vs. h+IFN with MO). *** or *** p<0.001 (CD3,⁺ cells: IL-2 with MO vs. h+IL-2 with MO).

FIG. 1B graphically depicts the percent of activation of CD4⁺ T-cells in the presence and absence of monocytes in response to IL-2 or IFN-α alone or with the H₂-receptor agonist, histamine. The parameters and symbols for this figure are the same as those in FIG. 1A.

FIG. 1C graphically depicts the percent of activation of CD8⁺/56⁻ T-cells in the presence and absence of monocytes in response to IL-2 or IFN-α alone or with the H2-receptor agonist, histamine. The parameters and symbols for this figure are the same as those in FIG. 1A.

FIG. 2 graphically depicts the results of FACS screenings of antibody labeled lymphocytes in histogram form. Lymphocytes and MO were incubated in microplates and treated with IL-2 and or histamine as described for FIG. 1A. Cells labeled with PE-conjugated monoclonal antibodies against CD3, and FITC-labeled monoclonal antibodies against CD69. Viable CD3,⁺ lymphocytes were gated and the relative fluorescence intensity and the percentage of cells stained with anti-CD69 was determined over 50,000 events. The individual graphs depict (A) lymphocytes+IL-2, (B) lymphocytes+MO+IL-2, (C) lymphocytes+histamine+IL-2, (D) lymphocytes+MO+IL-2+histamine.

FIG. 3 graphically depicts the percent of activation of CD3⁺ lymphocytes and CD56⁺ NK cells, in the presence of monocytes and treated with IFN-α (100 U/ml, filled bars), IL-2 (100 U/ml, open bars), culture medium (med), histamine (50 :M; h) and/or ranitidine (50 :M; ran) at 37° C. for 16 hours. Bars show CD69 expression and are representative of three similar experiments. CD3,⁺ T-cells and CD56⁺ NK cells were gated as described for FIG. 1A, incubated with MO and treated with IFN-α (100 U/ml, filled bars).

FIG. 4 graphically depicts the reversal of MO-induced inhibition of cytoking activation by catalase. Elutriated lymphocytes were incubated with MO and treated with IL-2 as described for FIG. 1A. Catalase was used at 0-200 U/ml. CD69 expression was monitored in CD3,⁺ T-cells by use of flow cytometry in gates comprising all viable lymphocytes. Data are the mean expression of CD69±s.e.m. in CD3,⁺ lymphocytes.

FIG. 5A graphically depicts the H₂-receptor agonist protection of T-cells and NK-cells from MO induced cell death. CD3,⁺ T-cells and CD56⁺ NK-cells were gated as described in the description of FIG. 1A. Cells were incubated with MO and treated with medium (med), IL-2 (100 U/ml) and IFN-α (100 U/ml; IFN), with (filled bars) or without (open bars) histamine (50 :M) at 37° C. for 16 hours. Cell death was measured by use of flow cytometry according to reduced forward scatter and increased right angle scatter. The data show the mean percentage of dead cells with respective phenotype÷s.e.m. obtained in experiments using cells from up to eleven blood donors. The open star (p<0.05) refers to a statistical comparison between CD3,⁺ T-cells and CD56⁺ NK-cells. The filled stars (*) refer to comparisons between cells incubated with and without histamine. * p<0.05, ** p<0.01. p<0.001.

FIG. 5B graphically depicts the H₂-receptor agonist protection of T-cells and NK-cells from MO induced cell death. CD4⁺ and CD8⁺/56⁻ T-cells were gated as described for FIG. 1A. Cells were incubated with MO and treated with medium (med), IL-2 (100 U/ml) and IFN-α (100 U/ml; IFN), with (filled bars) or without (open bars) histamine (50 :M) at 37° C. for 16 hours. Cell death was measured by use of flow cytometry according to reduced forward scatter and increased right angle scatter. The data show the mean percentage of dead cells with respective phenotype±s.e.m. obtained in experiments using cells from up to eleven blood donors. The open star (; p<0.05) refers to a statistical comparison between CD3,⁺ T-cells and CD56⁺ NK-cells. The filled stars (*) refer to comparisons between cells incubated with and without histamine. * p<0.05, ** p<0.01. *** p<0.001.

FIG. 6 graphically depicts the vaccine-induced proliferation of human mononuclear cells in vitro. A mixture of monocytes and T-cell enriched lymphoctes were treated with influenza vaccine (at indicated final dilutions) in the presence (filled bars) or absence (open bars) of histamine dihydrochloride (0.05 mM). Culture medium (med) was used as the control. The bars represent the mean counts per minute of 3H-TdR±s.e.m. of sextuplicate analysis performed in three healthy blood donors.

### DETAILED DESCRIPTION OF THE INVENTION

The present invention relates to methods of treating cancer or viral diseases in which histamine or an H₂-receptor agonist is administered alone or in conjunction with additional agents. The administration of these various agents results in the activation and protection of T-cells from the deleterious and inhibitory effects of monocytes/macrophages, as well as a stimulation of the anti-cancer and anti-viral properties of these cells. In addition, the administration of histamine in the presence of a vaccine composition results in an increase in lymphocyte proliferation in the presence of monocytes. The addition of other agents which are T-cell activation compounds that stimulate the cytotoxic activity of cytotoxic T-cells (CTLs), and other T-cell activities, preferably in a synergistic fashion with a H₂-receptor agonist are also contemplated. Representatives of such immunological stimulatory compounds include cytokines, peptides, flavonoids, vaccines, and vaccine adjuvants. Additional classes of agents usable with the methods of the present invention encompass chemotherapeutic and/or antiviral agents. The methods of the present invention also contemplate the use of radical oxygen metabolite scavengers in conjunction with the above mentioned compounds are also contemplated. The methods of the present invention are useful for treating neoplastic as well as viral disease.

In contemplating the treatment of individuals suffering from various neoplastic and viral diseases, the present invention seeks to stimulate and enhance cellmediated immunity to accomplish that end. Cell-mediated immunity (CMI) comprises the T lymphocyte-mediated immune response to a “foreign body.” The CMI response differs from the antibody-mediated humoral immunity in that the active agent in CMI is a T-cell rather than an antibody protein.

Cell-mediated immunity operates with cytotoxic T-cells or CTLs recognizing and destroying cells displaying “foreign” antigens on their surface. In the present invention a foreign body may be a neoplastic cell or a virus infected cell. As such, CMI functions to eliminate foreign cells from the body. For example, CMI would target cells infected with a virus, rather than to prevent the infection of the cell. Cell-mediated immunity, unlike humoral immunity which can be effective to prevent viral infection, remains the principal mechanism of defense against established viral infections. It is also pivotal in combating neoplastic disease. Therefore, the T-cell activity enhancing aspects of the present invention are uniquely suited to combat neoplastic and viral diseases.

As discussed above, the immune system contains a number of different cell types, each of which serve to protect the body for foreign invasion. Certain cells of the immune system produce radical oxygen metabolites (ROM) such as hydrogen peroxide, hypohalous acids, and hydroxyl radicals toward this goal. In previous observations, activation of human natural killer (NK)-cells in response to in vitro cytokine stimulation (e.g., IL-2 or IFN-α) is effectively inhibited by autologous monocytes/macrophages (MO). (For review see, Hellstrand, K., et al., Scand. J. Clin. Lab Invest. 57:193-202 (1997)). The inhibitory signal is conveyed by hydrogen peroxide or other reactiveoxygen metabolites (ROM) generated by MO. (See Hellstrand, K., et al., J. Immunol., 153: 4940-4947 (1994); Hansson, M., et al., J. Immunol. 156:42-47 (1996)). Addition of hydrogen peroxide scavengers which reduce the concentration of hydrogen peroxide and/or the addition of compounds which inhibit the release of hydrogen peroxide, such as histamine or H₂-receptor agonists, both have been shown to remove the inhibitory effects of MO. Id.

T-cells are considered important effector cells responsible for the antitumor properties of various cytokines such as IFN-α and IL-2, observed in experimental tumor models and in human neoplastic disease. (Sabzevari, H., et al., Cancer Res. 53: 4933-4937, (1993); Hakansson, A., et al., Br. J. Cancer, 74: 670-676, (1996); Wersall and Mellstedt, Med. Oncol., 12: 69-77, (1995)). The present invention relates, in part, to methods where compounds which reduce the concentration of ROM are used in conjunction with one or more T-cell activation compounds that result in T-cell activation or stimulation. The present invention, through the administration of ROM affecting compounds, T-cell activating compounds, and/or anticancer and antiviral compounds, provides methods to treat neoplastic disorders as well as viral infections by increasing the number and specific activity of T-cells.

A number of T-cell activation compounds are known in the art to activate and stimulate T-cell activity. The dosing, routes of administration and protocols for the use and administration of these materials can be the conventional ones, well known in the art. Generally, interleukins, cytokines and flavonoids have been shown to stimulate T-cell activity. Examples of suitable compounds are selected from the group consisting of IL-1, IL-2, IL-12, IL-15, IFN-α, IFN-β, IFN-γ and flavone acetic acid, xanthenone-4-acetic acid, and analogues or derivatives thereto.

Certain vaccines and vaccine adjuvants may also be considered T-cell activating compounds. Compounds contemplated here include a number of vaccines and vaccine adjuvants that assist administered antigens to induce rapid, potent, and long-lasting T cell mediated immune responses, from immunized or vaccinated individuals. Illustrative vaccines include influenza vaccines, human immunodeficiency virus vaccines, Salmonella enteritidis vaccines, hepatitis B vaccines, Boretella bronchiseptica vaccines, and tuberculosis vaccines, as well as various anticancer therapeutic vaccines such as allogeneic cancer and autologous cancer vaccines which are known in the art.

The present invention is also directed toward the use of a variety of vaccine adjuvants. Such agents including bacillus Calmette-Guerin (BCG), pertussis toxin (PT), cholera toxin (CT), E. coli heat-labile toxin (LT), mycobacterial 71-kDa cell wall associated protein, the vaccine adjuvant oil-in-water microemulsion MF59, microparticles prepared from the biodegradable polymers poly(lactide-co-glycolides) (PLG), immune stimulating complexes (iscoms) which are 30-40 nm cage-like structures, (which consist of glycoside molecules of the adjuvant Quil A, cholesterol and phospholipids in which antigen can be integrated), as well as other suitable compounds and compositions known in the art. Such compounds may be administered in amounts sufficient to elicit an effective immune response from an immunized individual.

The present invention contemplates and discloses a number of different T-cell activating compounds. These compounds may be used to form T-cell activating compositions that may be administered as a step of the present invention to achieve the activation of a patient's T-cells. The present invention contemplates the use of the terms T-cell activating compound and T-cell activation compositions as interchangeable. The dosing, routes of administration and protocols for the use and administration of these materials can be the conventional ones, well known in the art.

H₂-receptor agonists, histamine and other compounds with H₂-receptor agonist activity that are suitable for use in the present invention are known in the art. Examples of suitable compounds include compounds with a chemical structure resembling that of histamine or serotonin, yet do not negatively affect H₂-receptor activities. Suitable compounds are selected from the group consisting histamine, dimaprit, clonidine, tolazoline, impromadine, 4-methylhistamine, betazole, histamine congeners, H₂-receptor agonists, 8-OHDPAT, ALK-3, BMY 7378, NAN 190, lisuride, d-LSD, flesoxinan, DHE, MDL 72832, 5-CT, DP-5-CT, ipsapirone, WB 4101, ergotamine, buspirone, metergoline, spiroxatrine, PAPP, SDZ (−) 21009, and butotenine.

A variety of hydrogen peroxide (H₂O₂) scavengers effective to catalyze the decomposition of intercellular H₂O₂ are also known in the art. Suitable compounds are selected from the group consisting of catalase, glutathione peroxidase, ascorbate peroxidase, vitamin E, selen, glutathion, and ascorbate.

Administration of the compounds discussed above can be practiced in vitro or in vivo. When practiced in vitro, any sterile, non-toxic route of administration may be used. When practiced in vivo, administration of the compounds discussed above may be achieved advantageously by subcutaneous, intravenous, intramuscular, intraocular, oral, transmucosal, or transdermal routes, for example by injection or by means of a controlled release mechanism. Examples of controlled release mechanisms include polymers, gels, microspheres, liposomes, tablets, capsules, suppositories, pumps, syringes, ocular inserts, transdermal formulations, lotions, creams, transnasal sprays, hydrophilic gums, microcapsules, inhalants, and colloidal drug delivery systems.

The compounds of the present invention are administered in a pharmaceutically acceptable form and in substantially non-toxic quantities. A variety of forms of the compounds administered are contemplated by the present invention. The compounds may be administered in water with or without a surfactant such as hydroxypropyl cellulose. Dispersions are also contemplated, such as those utilizing glycerol, liquid polyethylene glycols, and oils. Antimicrobial compounds may also be added to the preparations. Injectable preparations may include sterile aqueous solutions or dispersions and powders which may be diluted or suspended in a sterile environment prior to use. Carriers such as solvents or dispersion media contain water, ethanol polyols, vegetable oils and the like may also be added to the compounds of the present invention. Coatings such as lecithins and surfactants may be used to maintain the proper fluidity of the composition. Isotonic agents such as sugars or sodium chloride may be added, as well as products intended to delay absorption of the active compounds such as aluminum monostearate and gelatin. Sterile injectable solutions are prepared according to methods well known to those of skill in the art and can be filtered prior to storage and/or use. Sterile powders may be vacuum or freeze dried from a solution or suspension. Sustained-release preparations and formulations are also contemplated by the present invention. Any material used in the composition of the present invention should be pharmaceutically acceptable and substantially non-toxic in the amounts employed.

Although in some of the experiments that follow the compounds are used at a single concentration, it should be understood that in the clinical setting, the compounds may be administered in multiple doses over prolonged periods of time. Typically, the compounds may be administered for periods up to about one week, and even for extended periods longer than one month or one year. In some instances, administration of the compounds may be discontinued and then resumed at a later time. A daily dose of the compounds may be administered in several doses, or it may be given as a single dose.

In addition, the compounds of the present invention can be administered separately or as a single composition (combined). If administered separately, the compounds should be given in a temporally proximate manner, e.g., within a twenty-four hour period, such that the activation of T-cells by the cytokine or other compound is enhanced. More particularly, the compounds may be given within 1 hour of each other. The administration can be by either local or by systemic injection or infusion. Other methods of administration may also be suitable.

The present invention also contemplates combinations of T-cell activation compounds with T-cell activating or stimulating properties, combinations of hydrogen peroxide production or release inhibiting compounds, combinations of hydrogen peroxide scavenging compounds, combinations of anticancer compounds, and combinations of antiviral compounds. The dosing, routes of administration and protocols for the use and administration of these materials can be the conventional ones, well known in the art. For example, IL-2 and IL-12 could be combined to activate a population of T-cells. Alternatively, a vaccine or an adjuvant could be used to activate a population of T-cells. Another example would be the combination of a H₂-receptor agonist such as dimaprit (SK&F, Hertfordshire, England) with histamine to inhibit the production or release of hydrogen peroxide from monocytes during a treatment regime. Combinations of various hydrogen peroxide compounds such as catalase and ascorbate peroxidase for example, are also contemplated. The present invention further contemplates using combinations of all of the various compounds discussed above to prepare an effective means to stimulate T-cells against neoplastic and/or viral disease.

All compound preparations may be provided in dosage unit forms for uniform dosage and ease of administration. Each dosage unit form contains a predetermined quantity of active ingredient calculated to produce a desired effect in association with an amount of pharmaceutically acceptable carrier. Such a dosage would therefore define an effective amount of a particular compound.

A preferred compound dosage range can be determined using techniques known to those having ordinary skill in the art. IL-2, IL-12 or IL-15 can be administered in an amount of from about 1,000 to about 600,000 U/kg/day (18 MIU/m²/day or 1 mg/m²/day); more preferably, the amount is from about 3,000 to about 200,000 U/kg/day, and even more preferably, the amount is from about 5,000 to about 10,000 U/kg/day.

IFN-α, IFN-β, and IFN-γ can also be administered in an amount of from about 1,000 to about 600,000 U/kg/day; more preferably, the amount is from about 3,000 to about 200,000 U/kg/day, and even more preferably, the amount is from about 10,000 to about 100,000 U/kg/day.

Flavonoid compounds can be administered in an amount of from about 1 to about 100,000 mg/day; more preferable, the amount is from about 5 to about 10,000 mg/day, and even more preferably, the amount is from about 50 to about 1,000 mg/day.

Commonly used doses for the compounds of the present invention fall within the ranges listed herein. For example, IL-2 is commonly used alone in doses of about 300,000 U/kg/day. IFN-α is commonly used at 45,000 U/kg/day. IL-12 has been used in clinical trials at doses of 0.5-1.5 μg/kg/day. Motzer, et al., Clin. Cancer Res. 4(5):1183-1191 (1998). IL-1 beta has been used at 0.005 to 0.2 μg/kg/day in cancer patients. Triozzi, et al., J. Clin. Oncol. 13(2):482-489 (1995). L-15 has been used in rates in doses of 25-400 μg/kg/day. Cao, et al., Cancer Res 58(8):1695-1699 (1998).

Vaccines and vaccine adjuvants can be administered in amounts appropriate to those individual compounds to activate T-cells. Appropriate doses for each can readily be determined by techniques well known to those of ordinary skill in the art. Such a determination will be based, in part, on the tolerability and efficacy of a particular dose using techniques similar to those used to determine proper chemotherapeutic doses.

Compounds effective to inhibit the release or formation of intercellular hydrogen peroxide, or scavengers of hydrogen peroxide, can be administered in an effective amount from about 0.05 to about 10 mg/day; more preferable, the amount is from about 0.1 to about 8 mg/day, and even more preferably, the amount is from about 0.5 to about 5 mg/day. Alternatively, these compounds may be administered from 1 to 100 micrograms per kilogram of patient body weight (1 to 100 μg/kg). However, in each case, the dose depends on the activity of the administered compound. The foregoing doses are appropriate and effective for histamine, H₂-receptor agonists, other intercellular H₂O₂ production or release inhibitors or H₂O₂ scavengers. Appropriate doses for any particular host can be readily determined by empirical techniques well known to those of ordinary skill in the art.

The present invention contemplates identifying a patient in need of enhanced T-cell activity and increasing that patient's circulating blood histamine or H₂-receptor agonist concentration to an optimum, beneficial, therapeutic level so as to more efficiently stimulate T-cell activity. Such a level may be achieved through repeated injections of the compounds of the present invention in the course of a day, during a period of treatment.

Subjects suffering from cancer often exhibit decreased levels of circulating blood histamine. (Burtin et al. Decreased blood histamine levels in subjects with solid malignant tumors, Br. J. Cancer 47: 367-372 (1983)). Thus, the elevation of blood histamine concentrations to beneficial levels finds ready application to cancer and antiviral treatments based on synergistic effects between histamine and agents which enhance cytotoxic effector cell mediated cytotoxicity. In such protocols, the activity of T-cells is enhanced. For example, the cytotoxic activity of cytotoxic T lymphocytes (CTLs) is enhanced by combining the administration of a H₂-receptor agonist such as histamine to increase circulating histamine to a beneficial level sufficient to augment the activity of an agent which acts in synergy with a H₂-receptor agonist to increase cytotoxicity with the administration of the agent.

In one embodiment of the present invention, beneficial levels of circulating blood H₂-receptor agonist are obtained by administering a H₂-receptor agonist at a dosage of 0.05 to 10 mg/day. In a another embodiment, beneficial blood levels of H₂-receptor agonists are administered at 1 to 100 microgram per kilogram of patient body weight (1 to 100 μg/kg). In a another embodiment, the H₂-receptor agonist is administered over a treatment period of 1 to 4 weeks with injections occurring as frequently as several times daily, over a period of up to 52 weeks. In still another embodiment, the H₂-receptor agonist is administered for a period of 1-2 weeks, with multiple injections occurring as frequently as several times daily. This administration can be repeated every few weeks over a time period of up to 52 weeks, or longer. Additionally, the frequency of administration may be varied depending on the patient's tolerance of the treatment and the success of the treatment. For example, the administrations may occur three times per week, or even daily, for a period of up to 24 months.

One embodiment the present invention contemplates utility with respect to the treatment of various cancers or neoplastic diseases. Malignancies against which the present invention may be directed include, but are not limited to, primary and metastatic malignant tumor disease, hematological malignancies such as acute and chronic myelogenous leukemia, acute and chronic lymphatic leukemia, multiple myeloma, Waldenstroms Macroglobulinemia, hairy cell leukemia, myelodysplastic syndrome, polycytaemia vera, and essential thrombocytosis.

The method of the present invention may also be utilized alone or in combination with other anticancer therapies. When used in combination with a chemotherapeutic regime, the H₂-receptor agonist and the T-cell activating compound are administered with a chemotherapeutic agent or agents. The dosing, routes of administration and protocols for the use and administration of these materials can be the conventional ones, well known in the art. Representative compounds used in cancer therapy include cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide. Procedures for employing these compounds against malignancies are well established. In addition, other cancer therapy compounds may also be utilized with the present invention.

The present invention contemplates treatment of a variety of viral diseases. The following are merely examples of some of the viral diseases against which the present invention is effective. There are a number of herpetic diseases caused by herpes simplex or herpes zoster viruses including herpes facialis, herpes genitalis, herpes labialis, herpes praeputialis, herpes progenitalis, herpes menstrualis, herpetic keratitis, herpes encephalitis, herpes zoster ophthalmicus, and shingles. The present invention is effective as a treatment against each of these diseases.

Another aspect of the shows the present invention to be effective against viruses that cause diseases of the enteric tract such as rotavirus mediated disease.

In another aspect, the present invention is effective against various blood based infections. For example, yellow fever, dengue, ebola, Crimean-Congo hemorrhagic fever, hanta virus disease, mononucleosis, and HIV/AIDS.

Another aspect of the present invention is directed toward various hepatitis causing viruses. A representative group of these viruses includes hepatitis A virus, hepatitis B virus, hepatitis C virus, hepatitis D virus, and hepatitis E virus.

In still another aspect, the present invention is effective against respiratory tract diseases caused by viral infections. Examples include: rhinovirus infection (common cold), mumps, rubella, varicella, influenza B, respiratory syncytial virus infection, measles, acute febrile pharyngitis, pharyngoconjunctival fever, and acute respiratory disease.

Another aspect of the present invention contemplates treatment for various cancer linked viruses, including: adult T-cell leukemia/lymphoma (HTLVs), nasopharyngeal carcinomas, Burkitt's lymphoma (EBV), cervical carcinomas, hepatocellular carcinomas.

In still a further aspect, the present invention is useful in the treatment of viral-meditated encephalitis, including: St. Louis encephalitis, Western encephalitis, and ticks.

The methods of the present invention may also be utilized alone or in combination with other antiviral therapies. When used in combination with an antiviral chemotherapeutic regime, the H₂-receptor agonist and the T-cell activating compound are administered with an antiviral chemotherapeutic agent or agents. The dosing, routes of administration and protocols for the use and administration of these materials can be the conventional ones, well known in the art. Representative compounds used in antiviral chemotherapy include idoxuridine, trifluorothymidine, adenine arabinoside, acycloguanosine, bromovinyldeoxyuridine, ribavirin, trisodium phosphophonoformate, amantadine, rimantadine, (S)-9-(2,3-Dihydroxypropyl)-adenine, 4′,6-dichloroflavan, AZT, 3′(-azido-3′-deoxythymidine), ganciclovir, didanosine (2′,3′-dideoxyinosine or ddI), zalcitabine (2′,3′-dideoxycytidine or denosine (ddA), nevirapine, inhibitors of the HIV protease, and other viral rs. The present invention also contemplates using a combination of anticancer and antiviral agents in conjunction with the administration of a H2-receptor agonist and/or an ROM scavenger.

Although not intended to limit the present invention, it is contemplated that the methods of the present invention augment T-cell activity by altering the mechanics of antigen presentation. One theory provides that monocytes/macrophages that are also antigen presenting cells (APC) are inhibited from presenting antigens to T-cells. This inhibition might result from MO metabolic pathways dedicated to the generation of ROM that inhibit MO antigen presenting metabolic pathways, producing mutually exclusive antigen presenting or ROM producing states in MO populations. A result of the inhibition of MO antigen presentation is that T-cell populations would remain dormant in the absence of presented antigen and in the presence of ROM.

Under this theory, administration of ROM production and release inhibiting compounds, such as histamine, acts to increase T-cell activity by increasing antigen presentation. Monocytes producing ROM may have a molecular switch thrown in the present of beneficial concentrations of histamine that results in a down regulation of ROM production. In the mutually exclusive metabolic state hyposized above, the down regulation of ROM production results in a subsequent increase in antigen presentation pathways and thus antigen presentation. Accordingly, administration of histamine in the presence of an antigen based T-cell activator, like a vaccine, would serve to increase T-cell activity by decreasing ROM production and increasing antigen presentation.

In an alternative theory, the administration of a ROM production and release inhibiting compounds, results in an increase T-cell activity by removing ROM induced T-cell inhibition.

The examples discussed below apply the teachings of the present invention and show that monocytes/macrophages (MO), and particularly MO-derived reactive oxygen metabolites (ROMs), effectively suppress the activation of human T-cells in response to the in vitro administration of T-cell activation compounds such IFN-α or IL-2. Furthermore, it is shown that the addition of a H₂-receptor agonist and a H₂O₂ confers protection to T-cells when added to a mixture of lymphocytes and MO.

To determine the effect of the various compounds of the present invention on a population of T-cells, the expression of the CD69 (Leu-23) antigen, an early activation antigen that is inducibly expressed on the surface of mature human T-cells was studied. The observed results show that cytokine-induced activation of T-cells, as reflected by the appearance of CD69 after incubation with representative cytokines such as IL-2 or IFN-α, was profoundly inhibited by MO in the absence of a H₂-receptor agonist or a H₂O₂ scavenger. However, addition of these compounds effectively reversed the observed inhibitory effects of MO. Additional work was performed to study the effect of histamine on the proliferative response of human lymphocytes to a polyvalent vaccine against influenza virus in vitro. The administration of histamine in these experiments was shown to elevate lymphocyte proliferation in presence of antigen and monocytes.

### EXAMPLES

The methods of the present invention may be used to enhance the activation and protection of T-cell populations using various T-cell activation compounds that result in T-cell stimulation and/or activation, H₂-receptor agonists, and H₂O₂ scavengers and inhibitors. To demonstrate the activation and protection characteristics of these compounds, lymphocytes (including T-cells) and monocytes were isolated from donated blood and examined for the activation characteristics when exposed various T-cell activating compounds, such as IL-2 and/or IFN-α, vaccines, vaccine adjuvants or other immunological stimulator compounds, various H₂-receptor agonists, such as histamine, and various H₂O₂ scavengers, such as catalase.

To study the activation characteristics of T-cells in the presence and absence of MO, T-cell activation compounds, H₂-receptor agonists, and H₂O₂ scavengers, peripheral venous blood was obtained as freshly prepared leukopacks from healthy blood donors at the Blood Centre, Sahlgren's Hospital, Göteborg, Sweden. The blood (65 ml) was mixed with 92.5 ml Iscove's medium, 35 ml 6% Dextran (Kabi Pharmacia, Stockholm, Sweden) and 7.5 ml acid citrate dextrose (ACD) (Baxter, Deerfield, Ill.). After incubation for 15 minutes at room temperature, the supernatant was carefully layered onto Ficoll-Hypaque (Lymphoprep, Myegaard, Norway). Mononuclear cells (MNC) were collected at the interface after centrifugation at 380 g for 15 minutes at room temperature, washed twice in PBS and resuspended in Iscove's medium supplemented with 10% human AB⁺ serum. During all further separation of cells, the cell suspensions were kept in siliconized test tubes (Vacuette, Greiner, Stockholm).

The MNC were further separated into lymphocyte and monocyte (MO) populations using the counter-current centrifugal elutriation (CCE) technique originally described by Yasaka and co-workers (Yasaka, T. et al., J. Immunol., 127:1515) with modifications as described in Hansson, M., et al. (J. Immunol., 156: 42 (1996); hereby incorporated by reference). Briefly, the MNC were resuspended in elutration buffer containing 0.05% BSA and 0.015% EDTA in buffered NaCl and fed into a Beckman J2-21 ultracentrifuge with a JE-6B rotor at 2100 rpm. A fraction with >90% MO was obtained at a flow rate of 18 ml/min. A lymphocyte fraction enriched for NK-cells (CD3⁻/56⁺ phenotype) and T-cells (CD3⁺/56⁻) was recovered at flow rates of 14-15 ml/min. This fraction contained <3% MO and consisted of CD3,⁻/56⁺ NK-cells (45-50%), CD3,⁺/56⁻ T-cells (35-40%), CD3,⁻/56⁻ cells (5-10%), and CD3,⁺/56⁺ cells (1-5%), as judged by flow cytometry. In some experiments, dynabeads (Dynal A/S, Oslo, Norway) coated with anti-CD56 were used to obtain purified lymphocyte preparations of T-cells, as described in detail by Hansson, M., et al., incorporated above.

Following fractionation, the lymphocyte mixture of T-cells and NK cells was exposed to the various experimental conditions described below and assayed for activation using the appearance of certain cell surface proteins as indicia of activation.

Lymphocytes are identifiable by certain proteins which reside on the cell surface. Different cell surface proteins reside on different classes of lymphocytes and lymphocytes in different stages of activation. These proteins have been grouped into CD classes or “clusters of differentiation” and may serve as markers for different types of cells. Labeled antibodies, specific for different cell surface proteins, that bind to the different CD markers may be used to identify the different types of T-cells and their respective states of activation.

In the experiments described below, CD3, CD4, CD8 and CD69 markers were used to identify the T-cells of interest. CD56 is a NK-cell marker. The CD3 group of antibodies is specific for a marker expressed on all peripheral T-cells. The CD4 group of antibodies is specific for a marker on class II MHC-restricted T-cells, also known as T helper cells. The CD8 group of antibodies recognize a marker on class I MHC-restricted T-cells, also known as CTLs or cytolytic T-cells. The CD69 group of antibodies recognizes activated T-cells and other activated immune cells. Finally, the CD56 groups recognizes a heterodimer on the surface of NK-cells.

Flow cytometry was used in the experiments described below to identify the various sub-populations of T-cells. Flow cytometry permits an investigator to examine a population of cells using a number of labeled probes to differentiate sub-populations within the larger whole. In these experiments, the CD3 marker was used to identify the subpopulation of T-cells and the CD4 and CD8 markers were used to further identify the subpopulation of T-cells into T helper cells and CTLs. The effects of MO exposure in the presence and absence of histamine and T-cell activation compounds were determined using the CD69 T-cell activation marker. The expression of the different markers was estimated in a lymphocyte gate using flow cytometry (as described in Hellstrand, K., et al. Cell. Immunol. 138: 44-54 (1991), and hereby incorporated by reference).

The following protocol was used in experiments reporting the detection of surface antigens of cell populations. One million cells were incubated with appropriate fluorescein isothiocynate (FITC) and phycoerythrin (PE) conjugated monoclonal antibodies (Becton & Dickinson, Stockholm, Sweden; 1:1/10⁶ cells), on ice for 30 minutes. The cells were washed twice in PBS and resuspended in 500:1 sterile filtered PBS and analyzed by use of flow cytometry on a FACSort with a Lysys II software program (Becton & Dickenson). Lymphocytes were gated on the basis of forward and right angle scatter. The flow rate was adjusted to <200 cells×s⁻ and at least 5×10³ cells were analyzed for each sample, if not otherwise stated.

### MO

In Example 1, isolateTo study the effect of MO on cytokine-induced lymphocyte activation and maturation the expression of CD69 on T-cells was monitored. Isolated peripheral blood lymphocytes were incubated with MO, T-cell activating compounds and/or H2-receptor agonists in Example 1. The results presented in this Example show that isolated T-cells are activated when exposed to various T-cell activating compounds.

### The Effect Of T-Cell Activating Compounds On CD69 Expression in Isolated Lymphocytes

### Example 1

Isolated peripheral blood lymphocytes (150,000 cells/well in a total volume of 0.2 ml) were incubated in microplates for 16 hours at 37° C. in the presence or absence of autologous MO. The cells were concomitantly treated with a T-cell activating compound such as IFN-α (100 U/ml) or IL-2 (100 U/ml), a H2-receptor agonist such as histamine (50:M) or culture medium (control). After completion of incubation, cells were washed twice and incubated with labeled monoclonal antibodies to the T-cell surface makers CD3, CD4, CD8, and CD69 or the NK-cell marker CD56 (purchased from Becton Dickinson, Stockholm, Sweden). The expression of the different antigens was estimated in a lymphocyte gate (set on the basis of forward and side scatter), and was compared in pure lymphocyte fractions (containing <3% MO) and in corresponding lymphocytes incubated with autologous MO. The following subsets were studied: CD3,⁺/4⁺, CD3,⁺/8⁺, and CD3,⁻/8⁻/56⁻, using flow cytometry.

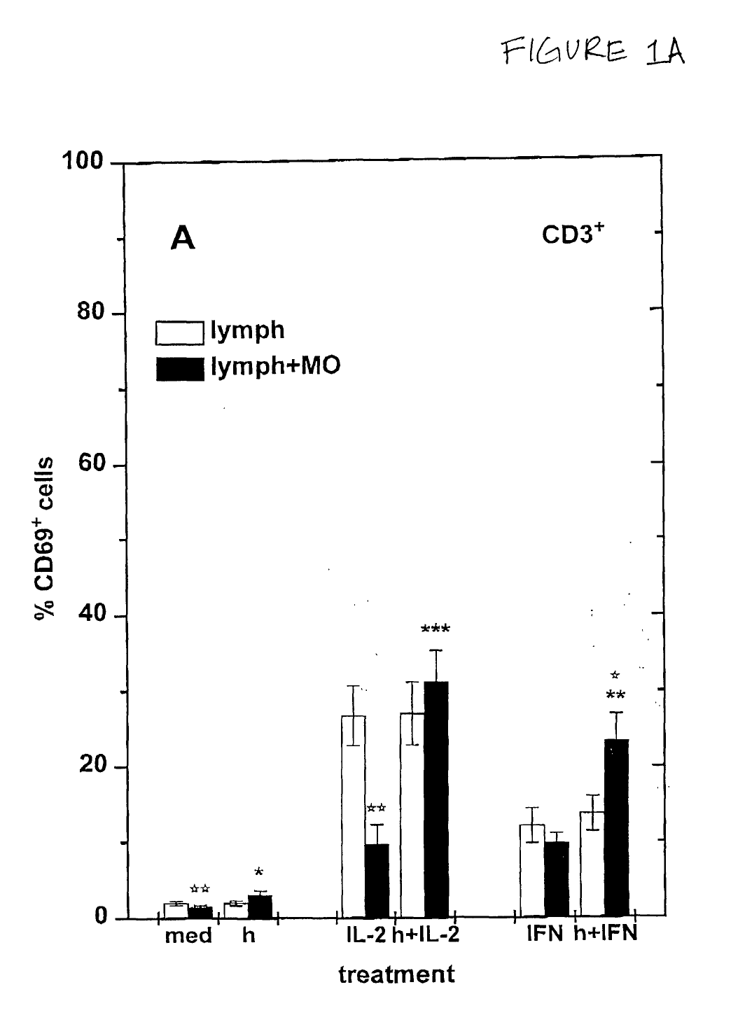

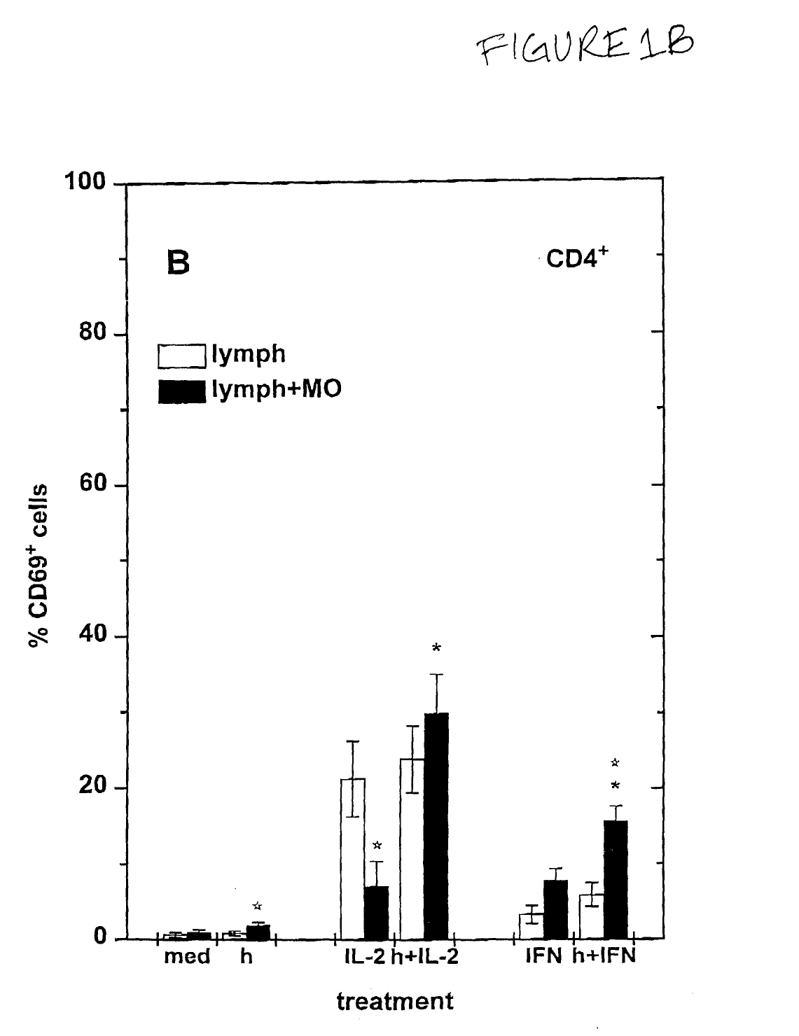

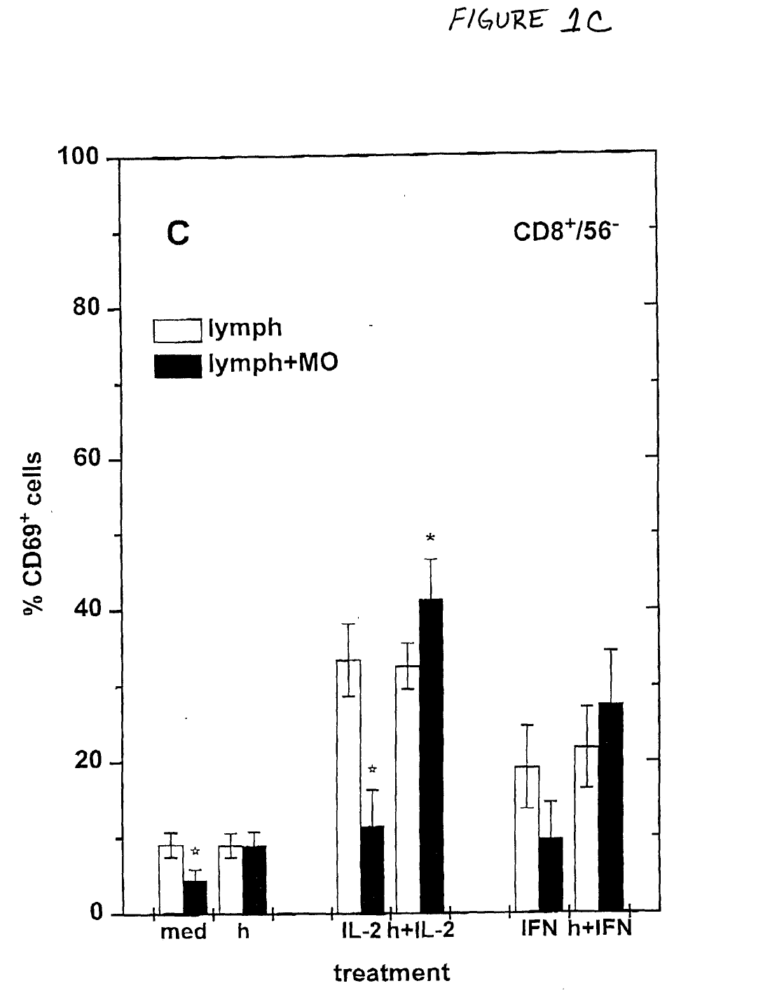

The cell surface expression of CD69 on unstimulated CD3e⁺ T-cells was low (˜2%). Approximately one fourth of CD3e⁺ cells acquired CD69 when treated with IL-2 (100 U/ml, 16 hours) in the absence of MO. The expression of CD69 when treated with IL-2 (100 U/ml, 16 hours) in the absence of MO. The expression of CD69 in unstimulated and IL2-activated CD3e⁺ cells was strongly reduced by the addition of MO (p<0.005). The induction of CD69 in CD3e⁺ cells in response to IFN-α was of lower magnitude (˜10%) than that induced by IL-2 and seemingly unchanged by the addition of MO (FIG. 1A). When CD4+ T-T-cells were studied, it was found that the constitutive expression of CD69 was low (<1%) and that the addition of IL-2 induced CD69 on approximately 20% of CD4⁺ cells, treated in the absence of MO. The acquisition of CD69 in response to IL-2 was inhibited by MO (p<0.05). A different pattern was observed for CD4⁺ cells activated by IFN-α. IFN-α was less effective than IL-2 in inducing CD69 in CD4⁺ cells incubated without MO (p<0.01), and a significantly higher IFN-α induced level of expression of CD69 on CD4⁺ cells was noted when MO were added (p<0.05; FIG. 1B).

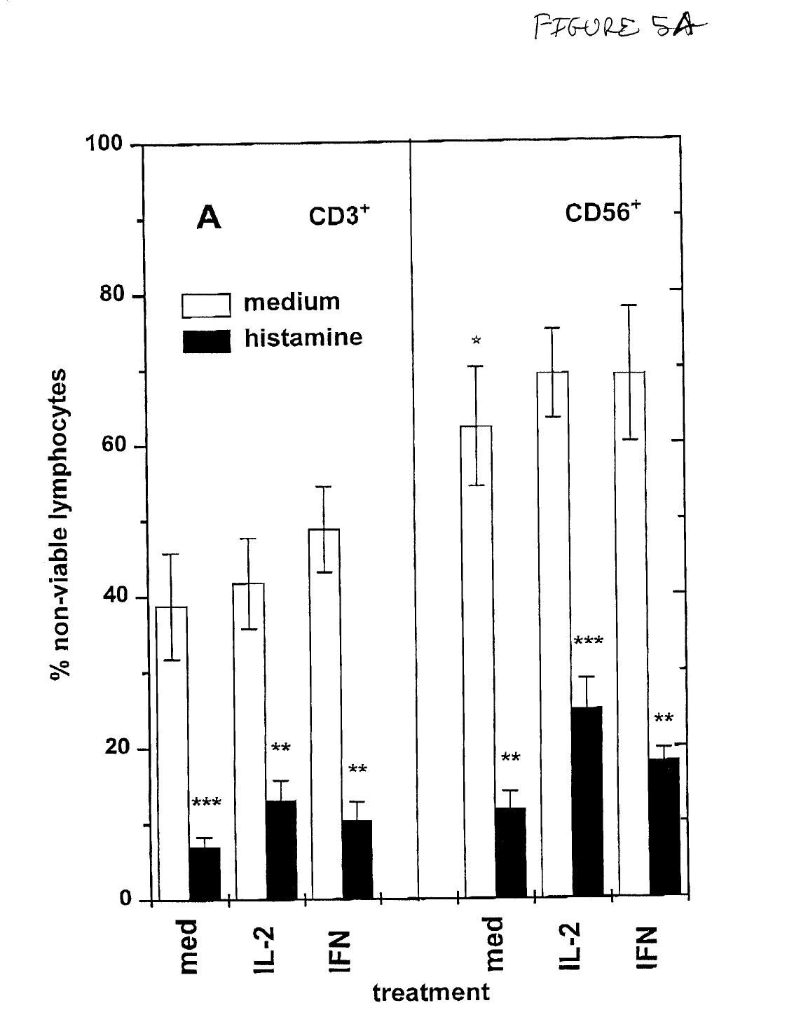

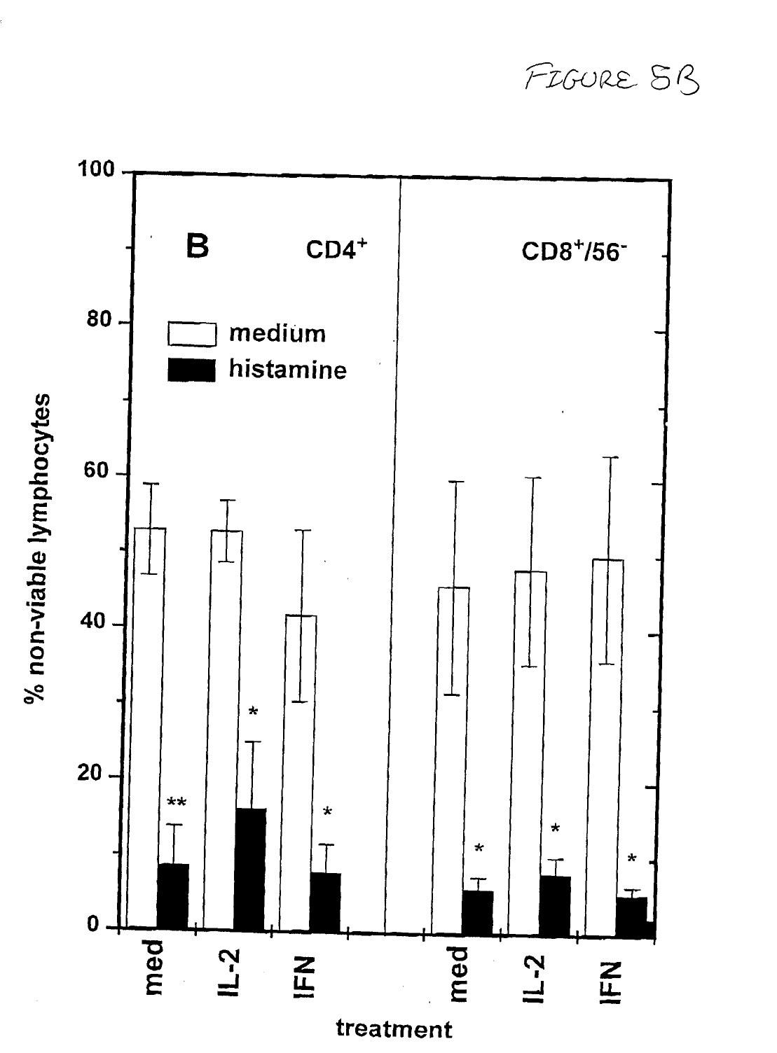

In studies of CD8⁺ T-cells, measures were taken to avoid contamination of the assayed cell population by CD8⁺ NK-cells. In a first set of experiments, CD8⁺ NK-cells were depleted by use of anti-CD56-coated beads. It was found that the constitutive expression of CD69 was significantly higher in CD8⁺ cells than in CD4⁺ cells (p<0.05). No significant qualitative differences between CD4⁺ cells and CD8⁺ T-cells as regards the induction of CD69 by IL-2 or the inhibition of the IL-2 response by MO were observed. A difference between CD4⁺ and CD8⁺ T-cells was that the addition of MO significantly suppressed (p<0.05) the constitutive expression of CD69 on CD8⁺ T-cells (FIG. 1C). Similar results were obtained in experiments in which three-color analysis of CD3e⁺/8⁺/56⁻ T-cells was performed. The data in FIG. 5 were obtained in experiments using a mixture of MO and lymphocytes.

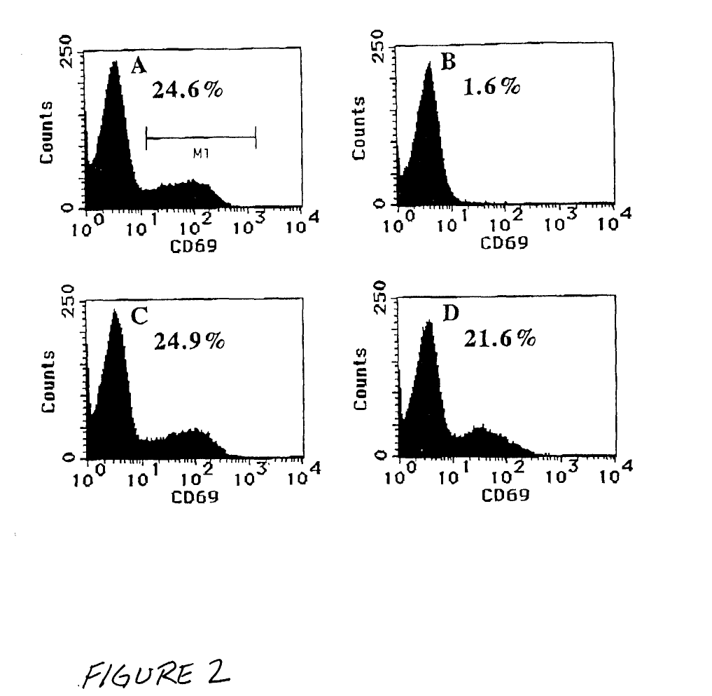

The presence of histamine did not significantly alter the expression of CD69 in either subset of non-stimulated or cytokine-activated T-cells incubated without MO. However, histamine counteracted the MO-induced inhibition of IL-2 induced acquisition of CD69 in T-cells; thus, histamine seemingly restored the expression of CD69 to the level observed in the absence of MO. FIG. 2 shows histograms of the IL-2-induced expression of CD69 in gated, viable CD3⁺ lymphocytes incubated with and without MO and treated with or without histamine. In CD3⁺ and CD4⁺ T-cells incubated with IFN-α, it was found that histamine enhanced the expression of CD69 to a significantly higher level when MO were present than that observed in absence of MO (FIGS. 1A and 1B). In contrast, the expression of CD69 in CD8⁺ cells activated with IFN-α was restored by histamine to the level observed in pure lymphocytes without a significant over-shoot (FIG. 1C).

The results from this example show that cytokine-induced activation of T-cells was strongly inhibited by autologous MO. Thus, in the subsets of lymphocytes tested, with the exception of IFN-α-treated CD4⁺ cells, acquisition of CD69 in response to IL-2 or IFN-α was markedly inhibited by MO.

### The Role of Radical Oxygen Metabolites in of Monocyte-Induced Inhibition of T-Cell Activation

To investigate the role of radical oxygen metabolites (ROM) in the monocyte-induced inhibition of T-cell activation, the roles of ROM, T-cell activating compounds, a H₂-receptor agonist, and a hydrogen peroxide scavenger were studied using isolated lymphocytes.

### Example 2

In this Example, elutriated lymphocytes were incubated with MO for 16 hours at 37° C. as described in Example 1. Catalase, a scavenger of hydrogen peroxide, was added at 10-200 U/ml. IL-2 was added at 100 U/ml. CD69 expression was monitored in the CD3,⁺ T-cells using flow cytometry in gates comprising all viable lymphocytes. Data are the mean expression of CD69±s.e.m. in CD3,⁺ lymphocytes.

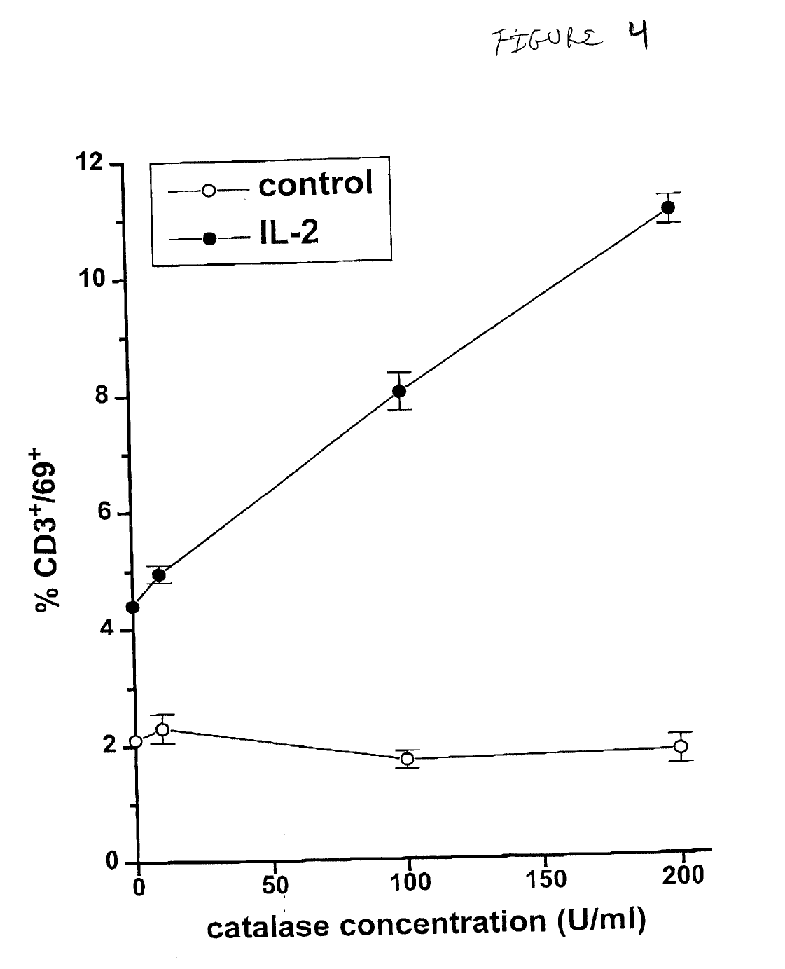

It was found that catalase significantly reversed the MO-induced inhibition of cytokine-induced CD69 expression (FIG. 4) but did not affect the induction of CD69 in either cell type in the absence of MO. Catalase alone over the concentration range of 0 to 200 U/ml had little effect of the percentage of CD3,⁺ cells expressing the CD69 marker. However, catalase in the presence of IL-2 had a much greater effect of CD69 expression. Specifically, the data show that only slightly greater than 4% of treated CD3,⁺ cells displayed the CD69 marker when treated with IL-2 alone and the absence of catalase. However, as the concentration of catalase increased from 0 to 200 U/ml the percentage of cells expressing the CD69 marker increased from the initial point to nearly 11%.

IL-2 stimulation was thus greatly increased in the presence of catalase and monocytes. These results suggest that it is the ROM produced by the MO which inhibits Tcell activation as measured by CD69 expression on CD3⁺ cells. The observed effect of catalase, a scavenger of ROM, reduced the inhibitory effect of MO on T-cell activation. The data shown in FIG. 4 indicates that the inhibition of T-cell activation may be reversed by scavenging ROM with catalase, and thus reducing the MO mediated inhibition of CD69 expression in response to stimulation by IL-2.

The Effect of H₂-Receptor Agonists and Antagonists On Cytokine Induced T-cell CD69 Expression

### Example 3

To investigate the effect of H₂-receptor agonists on MO-induced inhibition of T-cell activation measured by CD69 expression, CD3,⁺ T-cells were incubated with MO and treated with IFN-α (100 U/ml), IL-2 (100 U/ml), culture medium, a H₂-receptor agonist (histamine), and/or a H₂-receptor antagonists (ranitidine) at 37° C. for 16 hours.

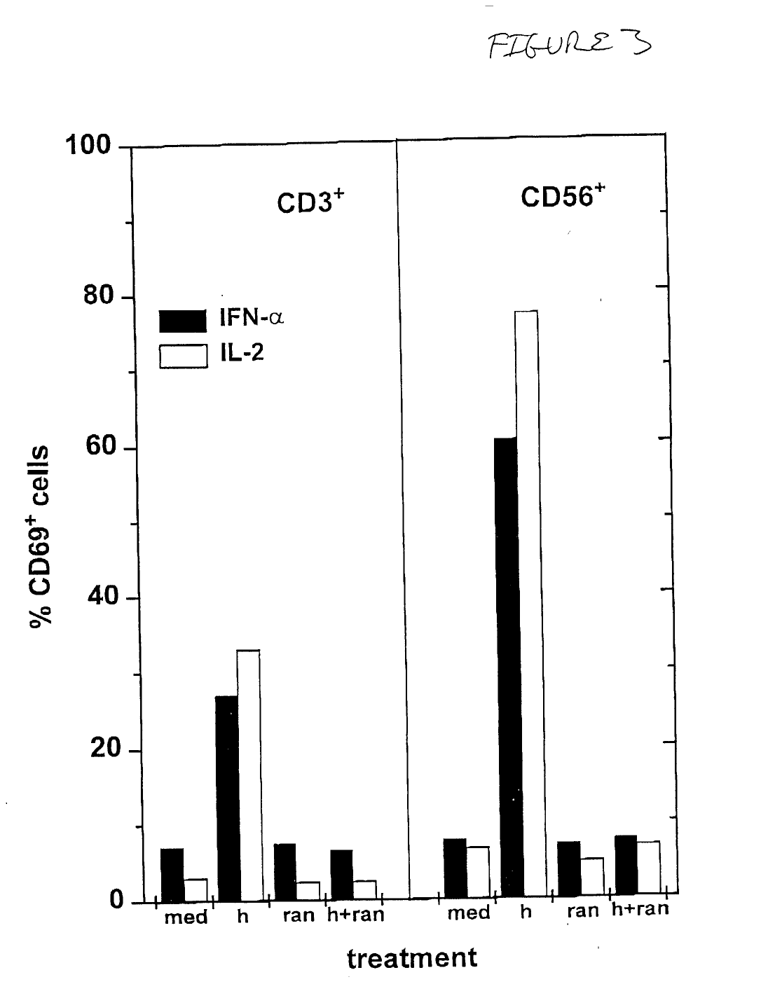

The effect of histamine on cytokine-induced expression of CD69 in T-cells was dose-dependent at final histamine concentrations of 0.1-50:M with an ED₅₀ of approximately 2:M. The histamine response was completely antagonized by ranitidine, an antagonist of H₂-type histamine receptors, used at equimolar or 10-fold lower concentrations. Smilar concentrations of AH20399AA, a chemical control to ranitidine in which the thioether group of ranitidine has been replaced by an ether thereby reducing its affinity for the H₂ receptor >50 fold, (Hellstrand, K., et al., J. Leukoc. Biol., 55:392 (1994)), did not block the histamine effect (FIG. 3 and data not shown).

The results from this Example show that the H₂-receptor agonist histamine was capable of specifically reversing the MO-mediated inhibition of T-cell activation as measured by CD69 expression. The specificity of this effect was demonstrated with the antagonist ranitidine.

### Histamine Protection of T-Cells from MO-Induced Apoptosis

### Example 4

In this Example, apoptotic morphology of lymphocytes exposed to MO was monitored by staining cells with a dye mix containing acridine orange (10:g/ml; Sigma) and ethidium bromide (10:g/ml; Sigma), both prepared in phosphate buffered saline. One microliter (1:1) of dye mix was added to 25:1 of cells suspension (1-2×10⁶/ml) in siliconized test tubes. Thereafter, 10:1 of the cell suspension was placed on a glass slide and immediately counted in a fluorescence microscope (Nikon) under times forty (×40) magnification with qualification of dead, living, apoptotic and non-apoptotic cells. (See Hellstrand, K., et al. J. Immunol., 153: 4940-4947 (1994); Hansson, M., et al., J. Immunol. 156:42-47 (1996)).

We have earlier demonstrated that human T-cells and NK-cells differ in their sensitivity to oxidative stress. Approximately 5-fold higher concentrations of exogenous hydrogen peroxide are required to induce apoptosis in CD3e⁺ T-cells than in CD56⁺ NK-cells. (See Hansson M., et al., supra). Cell death in lymphocytes was monitored by gating nonviable T-cells or NK-cells after exposure to MO, with and without histamine or catalase. A gate with reduced forward scatter and increased right angle scatter characteristic of apoptosis was employed in these studies. (See Hansson M., et al., supra; Mizgerd J. P., et al., J. Leukoc. Biol. 59:189 (1996); herein incorporated by reference; gate also described in Example 1). The cells were predominantly apoptotic, as revealed by conventional staining with acridine orange and ethidium bromide.

Exposure of lymphocytes to MO induced considerable cell death in lymphocytes. Thus, a large fraction of both T-cells and NK-cells acquired reduced forward scatter and increased right angle scatter after overnight incubation with autologous MO. When T- and NK-cell markers were investigated in the population of apoptotic lymphocytes, it was found that the frequency of NK-cells was significantly higher than T-cells. Thus, 62% of NK-cells and 39% of CD3,⁺ T-cells died after contact with MO, and this difference reached statistical significance (p<0.05; FIG. 5A). Similarly, 45-55% of CD4⁺ or CD8⁺/56⁻ cells died after contact with MO. The propensity of cell death was apparently similar in CD4⁺ cells and in CD8⁺ cells (FIG. 5B). The frequency of T- or NK-cells carrying CD69 was similar in dead and living lymphocytes, thus suggesting that induction of CD69 can occur also in cells prone to apoptosis.

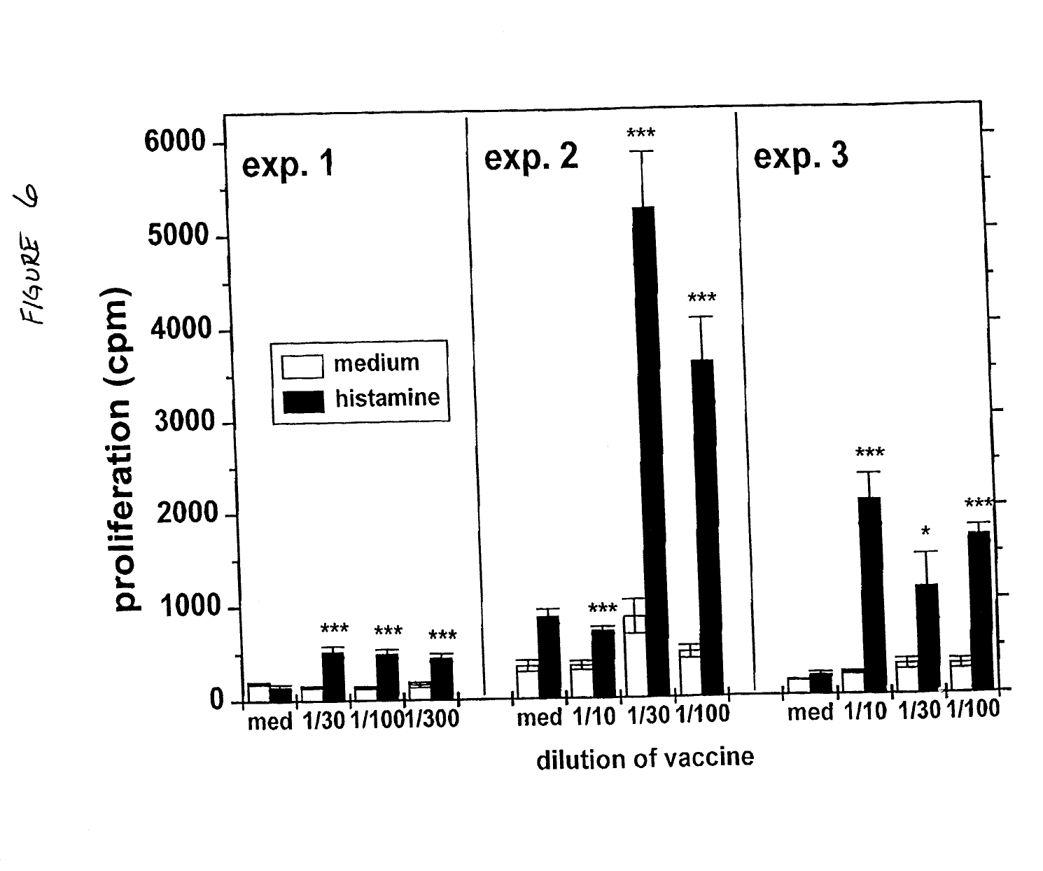

The results from this Example show that histamine significantly prevented MO-induced cell death by >80% in all subsets of T-cells and in NK-cells. The MO-induced cell death as well as the protection afforded by histamine was unaffected by concomitant treatment with IL-2 or IFN-α (FIGS. 6A and 6B). The effect of histamine on MO-induced cell death was mimicked by catalase and completely reversed by ranitidine, but not by AH20399AA at concentrations equimolar to 10-fold lower than histamine.

### Treatments Employing a Combination of a H₂-Receptor Agonist and a T-Cell Activation Compound

The increased blood H₂-receptor agonist levels discussed above find application in treatments of patients identified as being in need of enhanced T-cell activity, where CTL cytotoxicity is augmented through the synergistic effects of H₂-receptor agonist and an immunological stimulatory compound that enhances T-cell cytotoxicity or activity. As discussed above, one such enhancer of cytotoxicity is IL-2. Examples 5 and 6 describe methods of treatment in which beneficial level of a H₂-receptor agonist is achieved through the administration of histamine which augments the activity of IL-2.

### Example 5

Histamine, a H₂-receptor agonist, in a dose approximately 0.2 to 2.0 mg or 3-10 μg/kg, in a pharmaceutically acceptable form is injected subcutaneously in a sterile carrier solution into subjects in need of enhanced T-cell activity, in this case a patient having a malignancy. Concomitantly, IL-2, for example, human recombinant IL-2 (Proleukin®, Eurocetus), is administered subcutaneously or by continuous infusion of 27 μg/kg/day on days 1-5 and 8-12. This dose represents a total dose of IL-2 considerably lower than that administered by those of skill in the art.

The above procedure is repeated every 4-6 weeks until an objective regression of tumor disease is observed. The therapy may be continued even after a partial or complete response has been observed. In patients with complete responses, the therapy may be given with longer intervals between cycles.

The treatment may also include periodically boosting patient blood histamine levels by administering 0.2 to 2.0 mg or 3-10 μg/kg of histamine injected 1, 2, or more times per day over a period of one to two weeks at regular intervals, such as daily, biweekly, or weekly in order to establish blood histamine at a beneficial concentration.

### Example 6

Human recombinant 1L-2 (Proleukin), Eurocetus) is administered by subcutaneous injection or continuous infusion at a rate of 27 μg/kg/day on days 1-5 and 8-12 into patients in need of enhanced T-cell activity, in this case patients infected with herpes simplex virus (HSV) type 2. Injections of histamine at 0.2 to 2.0 mg or 3-10 μg/kg per injection in a pharmaceutically acceptable form are injected subcutaneously in a sterile carrier solution to establish a therapeutic blood histamine level.

The above procedure is repeated every 4-6 weeks until an objective regression of the disease is observed. The therapy may be continued even after a first, second or subsequent complete remission has been observed.

The treatment may also include periodically boosting patient blood histamine levels by administering 0.2 to 2.0 mg or 3-10 μg/kg of histamine injected 1, 2, or more times per day over a period of one to two weeks at regular intervals, such as daily, biweekly, or weekly in order to beneficialachieve a beneficial blood histamine concentration.

### Combination of H₂-Receptor Agonists and T-Cell Activating Compounds

Beneficial levels of circulating blood H₂-receptor agonists, such as histamine can also be employed in conjunction with treatments involving immunological stimulatory compounds that result in an enhancement of T-cell numbers, activity, or function. Example 7 describes how to administer such treatments.

### Example 7

Subjects in need of enhanced T-cell activity caused directly or indirectly by a neoplastic disease, and/or a viral infection such as hepatitis B (HBV), hepatitis C (HCV), human immunodeficiency virus (HIV), human papilloma virus (HPV) or herpes simplex virus (HSV) type 1 or 2, or other viral infections, are administered human recombinant IL-2 (Proleukin®, Eurocetus) by subcutaneous injection or by continuous infusion of 27 μg/kg/day on days 1-5 and 8-12. Additionally, subjects may also receive a daily dose of 6×10⁶ U interferon-α administered by a suitable route, such as subcutaneous injection. This treatment also includes administering 0.2 to 2.0 mg or 3-10 μg/kg of histamine injected 1, 2, or more times per day in conjunction with the administration of IL-2 and/or interferon-α.

The above procedure is repeated every 4-6 weeks until an objective regression of the tumor is observed, or until improvement in the viral infection occurs. The therapy may be continued even after a first, second, or subsequent complete remission has been observed. In patients with complete responses, the therapy may be given with longer intervals between cycles.

The treatment may also include periodically boosting patient blood histamine levels by administering 0.2 to 2.0 mg or 3-10 μg/kg of histamine injected 1, 2, or more times per day over a period of one to two weeks at regular intervals, such as daily, biweekly, or weekly in order to establish or maintain blood histamine at a beneficial concentration, e.g., at a concentration above 0.2 μmole/L.

Additionally, the frequency of interferon-α administration may be varied depending on the patient's tolerance of the treatment and the success of the treatment. For example, interferon may be administered three times per week, or even daily, for a period of up to 24 months. Those skilled in the art are familiar varying interferon treatments to achieve both beneficial results and patient comfort.

Combination of a H₂-receptor Agonist and Chemotherapeutic Agents

A H₂-receptor agonist may also be used in conjunction with chemotherapeutic agents to treat a neoplastic or viral disease. Typically, levels of circulating histamine decline during chemotherapy. Low levels of circulating histamine may result in the suppression of CTL cytotoxicity by monocytes. Thus, these patients are in need of enhanced T-cell activity. This monocyte mediated suppression may be eliminated by administration of a H₂-receptor agonist, like histamine, prior, during, following or throughout chemotherapy in order to increase the blood histamine concentration to a beneficial level.

Accordingly, the present invention contemplates the increase of circulating blood histamine levels in conjunction with chemotherapeutic agents. Additionally, the treatment may also include the administration of an immunological stimulator compound that results in T-cell activation, such as L-2, interferon-α and/or a vaccine or vaccine adjuvant.

Representative compounds used in cancer and antiviral therapies are described above. Other cancer and antiviral therapeutic compounds may also be utilized in the present invention. Similarly, malignancies and viral diseases against which the treatment of the present invention may be effective and thus may be directed are also described. It should be noted that the amounts, routs of administration and dosage protocols for these cancer and antiviral compounds used with the methods of the present invention may be those well known to those of skill in the art. The present invention is directed toward augmenting the efficacy of these compounds, and the therapeutic results of their use. Therefore, the conventional methodologies for their use, in conjunction with the compounds and methods of the present invention, are contemplated as sufficient to achieve a desired therapeutic effect.

A combination of histamine and IL-2 for activating NK cells has proven an effective combination with traditional chemotherapeutic methods in treating acute myelogenous leukemia. Brune and Hellstrand, Br. J. Haematology, 92:620-626 (1996). Procedures for using the H₂-receptor agonists of the present invention in combination with various chemotherapeutic and immunological stimulating agents such as IL-2 for stimulating T-cells are presented in Examples 8 through 10. It will be appreciated that beneficial levels of circulating histamine may also be employed in treatments using only chemotherapeutic agents or immunological stimulating agents.

### Example 8

Subjects with AML in first, second, subsequent or complete remission are treated in 21-day courses with IL-2 [35-50 μg (equivalent to 6.3-9×10⁵ IU) subcutaneously (s.c.). twice daily], repeated with three to six-week intermissions and continued until relapse. In cycle #1, patients receive three weeks of low dose chemotherapy consisting of 16 mg/m²/day cytarabine, and 40 mg/day thioguanine. Concomitantly, patients are injected subcutaneously with 0.2 to 2.0 mg or 3-10 μg/kg of a pharmaceutically acceptable form of a H₂-receptor agonist such as histamine to boost circulating histamine to a beneficial level twice daily (above 0.2 μmole/L). Histamine levels may be continually boosted to beneficial levels by administering histamine by injection at 0.2 to 2.0 mg or 3-10 μg/kg twice daily in a pharmaceutically acceptable form of a H₂-receptor agonist during the IL-2 treatment. Thereafter, the subjects are allowed to rest for three to six weeks.

After the rest period at the end of the first cycle (cycle #1), the second cycle (cycle #2) is initiated. Twice daily, injections of a pharmaceutically acceptable form of a H₂-receptor agonist in a sterile carrier solution are administered at 0.5 to 2.0 mg or 3-10 μg/kg subcutaneously. Cytarabine (16 mg/m²/day s.c.) and thioguanine (40 mg/day orally) are given for 21 days (or until the platelet count is <50×10⁹/1). In the middle week, patients receive 0.2 to 2.0 mg or 3-10 μg/kg per injection twice per day of a pharmaceutically acceptable form of histamine to boost circulating histamine to beneficial levels. At the end of the three week chemotherapy treatment, patients receive 0.2 to 2.0 mg or 3-10 μg/kg per injection twice daily of a pharmaceutically acceptable form of histamine for a week. Thereafter, patients receive interleukin-2 for three weeks. Patients are permitted to rest for three to six weeks.

Thereafter, cycle #3 is initiated. Cycle #3 is identical to cycle #2.

Alternatively, the treatment may also include periodically boosting patient blood histamine levels by administering 0.2 to 2.0 mg or 3-10 μg/kg of histamine injected 1, 2, or more times per day over a period of one to two weeks at regular intervals, such as daily, bi-weekly, or weekly in order to achieve a beneficial blood histamine concentration. Another alternative is to provide histamine in a depot or controlled release form.

### Example 9

Subjects having a malignancy, neoplastic disease, or viral infection implicating inadequate T-cell activity and caused by contagia such as hepatitis B, hepatitis C, human immunodeficiency virus (HIV), human papilloma virus (HPV) or herpes simplex virus (HSV) type 1 or 2 or other viruses, are administered 0.1-5.0 mg/day of a pharmaceutically acceptable form of histamine or another H₂-receptor agonist. The H₂-receptor agonist is administered for a period of one week up to 12 months above or in combination with antiviral compounds and/or T-cell activating agents.

The above procedure is repeated until an objective regression of the tumor is observed, or until improvement in the viral infection occurs. The therapy may be continued even after a partial or complete response has been observed. In patients with complete responses, the therapy may be given with longer intervals between cycles.

The treatment may also include periodically boosting patient blood histamine levels by administering 0.1 to 5.0 mg or 1-50 μg/kg of histamine injected 1, 2, or more times per day over a period of one to two weeks at regular intervals, such as daily, biweekly, or weekly in order to establish or maintain blood histamine at a beneficial concentration.

Histamine in a pharmaceutically acceptable form, such as a sterile carrier solution, can be injected subcutaneously 0.1-5.0 mg/injection, 1-4 times per day in order to increase circulating blood histamine to a beneficial level.

### Example 10

Subjects suffering from a malignancy or viral infection implicating inadequate T-cell activity caused by viruses such as hepatitis B, hepatitis C, human immunodeficiency virus (HIV), human papilloma virus (HPV) or herpes simplex virus (HSV) type 1 or 2, or other viruses, are administered 0.1 to 5.0 mg or 1-50 μg/kg per injection of a pharmaceutically acceptable form of histamine or another H₂-receptor agonist. Concurrently, an anticancer and/or an antiviral agent may be administered in conjunction with the a H₂-receptor agonist, using standard dosages, routes of administration, and protocols well known in the art.

The above procedure is repeated every 4-6 weeks until an objective regression of the tumor is observed, or until improvement in the viral infection occurs. The therapy may be continued even after a partial or complete response has been observed. In patients with complete responses, the therapy may be given with longer intervals between cycles.

Histamine in a pharmaceutically acceptable form, such as a sterile carrier solution, can be injected subcutaneously 0.1 to 5.0 mg or 1-50 μg/kg per injection, 1, 2, or more times per day over a period of one to two weeks at regular intervals, such as daily, biweekly, or weekly in order to achieve a beneficial blood histamine concentration.

### The Effect of Histamine on the Proliferative Response of Human Mononuclear Cells Challenged with a Polyvalent Vaccine Against the Influenza Virus

Induction of immunity by vaccination or infection includes a proliferative response of T-cells to antigens. The antigen-induced proliferation of lymphocytes requires monocytes or other accessory cells, which present antigen to lymphocytes in conjunction with major histocompatibility products. Also, monocytes provide accessory signals of importance for the proliferation of lymphocytes.

Histamine, a biogenic amine stored in circulating basophilic leukocytes and in tissue-bound mast cells, has been ascribed several regulatory effects on immune effector mechanisms. Reviewed in Beer et al., Adv. Immunol. 35:209-263 (1984). Histamine has been shown to reduce the proliferation of lymphocytes in response to lectins such as phytohemagglutinin and to bacterial toxins such as staphylococcal enterotoxin type A. Dohlsten et al., Cellular Immunology 109:65-74 (1987). These and other effects of histamine on lymphocyte function are mediated by H₂-type histamine receptors.

A limitation of the reports showing that histamine inhibits the proliferation of lymphocytes is that a low amount of monocytes was used (<10%). In several types of tissues, monocytes are present in higher amounts. For example, in solid tumors monocytes or monocytes-like cells are frequently found to be the predominant infiltrating mononuclear cell type. Alexander et al., Ann. NY Acad. Sci. 276:124-33 (1976).

To more adequately address the in vivo situation in, e.g., tumor tissue, the effects of histamine on antigen-induced proliferation of lymphocytes were studied in a mixture of lymphocytes and 50% monocytes in vitro. A prototypic polyvalent human influenza vaccine was used as the inducer of lymphocyte proliferation. The data unexpectedly show that histamine strongly enhances the proliferative response to this vaccine.

### Example 11

Peripheral venous blood samples were obtained and MNC were prepared as described above. The cells were further separated as described above, and a lymphocyte fraction enriched for T-cells(CD3⁺/56⁻) was recovered at flow rates of 13-14 ml/min. This fraction did not contain monocytes.

The T-cell enriched lymphocytes (0.9×10⁵ cells/well) were incubated in sextuplicate in microplates in a total volume of 150 μl in the presence or absence of monocytes (0.9×10⁵ cells/well). Histamine dihydrochloride (0.05 mM)(Sigma Chemicals, St. Louis, USA) or culture medium (control) was added at the onset of incubation at 37° C. for 72-96 hours. All wells received 15 μl of polyvalent influenza vaccine (Begrivac® , Hoechst; purchased from SBL Vaccine AB, Stockholm, Sweden) at various dilutions described below. To quantitate proliferation, cells were pulsed with ³H-methyl-thymidine (³H-TdR; specific activity 2 Ci/mole); New England Nuclear Corp.; 1 μCi/2×10⁵ cells) for 8 hours. The cells were collected on glass fiber filters with an automatic cell harvester. The amount of cellular incorporation of ³H-TdR was estimated by solid-phase scinitillography.

FIG. 6 shows the effects of histamine on the proliferation of T-cell enriched lymphocytes induced by influenza vaccine. A mixture of monocytes and T-cellocytes enriched lymphocytes was treated with influenza vaccine (at indicated dilutions) in the presence (filled bars) or absence (open bars) of histamine dihydrochloride (0.05 mM). Culture medium (med) was used as the control. The data represent in the bars are the mean counts per minute of 3H-TdR±s.e.m. of sextuplicate analysis performed in three healthy blood donors and reflect DNA synthesis as a measure of cellular proliferation. Results obtained using cells from three different healthy blood donors (experiments 1-3) are shown.

The data shown show histamine has a profound effect on the proliferation response. In control cells, i.e., cells not treated with the vaccine, histamine alone slightly augmented proliferation. Similarly, the vaccine alone only weakly induced proliferation. In contrast, histamine strongly potentiated vaccine-induced proliferation at all dilutions of the vaccine studied. The effect of the combination of vaccine and histamine was significantly higher than that induced by vaccine alone (p<0.001 at final vaccine dilutions of 1/10, 1/30, 1/100, and 1/300 in experiments 1 and 3; p<0.05 at a vaccine dilution of 1/30 in experiment 3). Further, the proliferation of cells treated with vaccine and histamine was significantly higher (p<0.05−p<0.001) than that induced by histamine alone at vaccine dilutions of 1/10 (experiment 3), 1/30 (experiments 1, 2, and 3), 1/100 (experiments 1, 2, and 3), and 1/300 (experiment 1). The observed significant increase in cellular proliferation indicates that the combination of a vaccine and histamine results in an increased level of T-cell enriched lymphocyte proliferation.

### CONCLUSION

The data presented herein demonstrate that MO inhibit T-cell activation. MO inhibition of T-cell activation appears to be mediated by ROM formation. The experiments discussed above show that MO inhibition of T-cells may be reversed through the addition of a ROM formation inhibitor such as histamine, or a ROM scavenger such as catalase. These results suggest that T-cell activation may benefit from a down-regulation of MO inhibition.

The results above also show that CD3⁺ T-cells are refractory to cytokine stimulation in the presence of MO. The results also show that histamine almost completely counteracted the MO-induced prevention of cytokine-induced acquisition of CD69 in CD3⁺, CD4 and CD8⁺ T-cells. The positive effect of histamine on CD69 expression in the presence of MO suggest that therapeutic anticancer or antiviral regimes that target T-cells as effector cells would benefit from a down regulation of MO inhibition.

The experiments discussed above show that histamine, in combination with an immunological stimulatory compound that results in T-cell stimulation or activation, can substantially increase the levels of T-cell activation in response to the stimulating compound. These observations have clinical importance, since T-cells play such a key role in the immune system response to tumors and viral infections. From the results shown above it is clear that the relationship between H₂-receptor agonists and T-cell activating compounds may be exploited to increase the efficacy of therapeutic agents, such as antiviral and anticancer agents.

What is claimed is:

1. A method for enhancing T-cell proliferation, comprising:
administering an effective amount of a T-cell proliferation enhancing composition to a population of T-cells and monocytes, wherein the composition selected from the group consisting of a T-cell proliferation enhancing vaccine adjuvant, a T-cell proliferation enhancing vaccine, a T-cell proliferation enhancing peptide, a T-cell proliferation enhancing cytokine selected from the group consisting of IL-1, IL,-12, IL,-15, IFN-β, and IFN-γ, and a T-cell proliferation enhancing flavonoid; and
administering about 0.05 to about 10 mg per day of a compound that inhibits the production or release of intercellular reactive oxygen metabolites (ROM), which is selected from the group consisting of histamine, histamine dihydrochloride, serotonin, dimaprit, clonidine, tolazoline, impormadine, 4-methylhistamine, betazole, and a histamine congener.

2. The method of claim 1, wherein the vaccine adjuvant is selected from a compound from the group consisting of bacillus Calmette-Guerin (BCG), pertussis toxin (PT), cholera toxin (CT), E. coli heat-labile toxin (LT), mycobacterial 71-kDa cell wall associated protein, microemulsion MF59, microparticles of poly(lactide-co-glycolides)(PLG), and immune stimulating complexes (ISCOMS).

3. The method of claim 1, wherein said T-cell proliferation enhancing composition is a cytokine selected from the group consisting of IL-1, IL-12, IL-15, IFN-β, and IFN-γ and is administered in a daily dose of between 1,000 and 600,000 U/kg.

4. The method of claim 1, wherein said compound that inhibits the production or release of intercellular ROM is administered at about 0.05 to about 10 mg per dose.

5. The method of claim 1, wherein said compound that inhibits the production or release of intercellular ROM is administered at 1 to 100 μg/kg of patient weight per dose.

6. The method of claim 1 further comprising the step of administering an effective amount of a scavenger of intercellular hydrogen peroxide.

7. The method of claim 6, wherein the scavenger is selected from the group consisting of catalase, glutathione peroxidase, and ascorbate peroxidase.

8. The method of claim 6, wherein said hydrogen peroxide scavenger is administered in a dose of from about 0.05 to about 50 mg/day.

9. The method of claim 6, wherein said T-cell activating composition and said scavenger of intercellular hydrogen peroxide are administered separately.

10. The method of claim 1, further comprising the step of administering a chemotherapeutic agent to said patient.

11. The method of claim 10, wherein the chemotherapeutic agent comprises an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide.

12. The method of claim 10, wherein the steps of administering said T-cell activating composition, said compound that inhibits the production or release of intercellular reactive oxygen metabolites (ROM) and said chemotherapuetic agent are performed concomitantly.

13. The method of claim 1, wherein said reactive oxygen metabolites comprise hydrogen peroxide.

14. The method of claim 6, wherein the administration of said T-cell activating compound and the administration of said scavenger of intercellular hydrogen peroxide are performed within 24 hours of each other.

15. The method of claim 1, wherein the administration of said T-cell activating composition and the administration of said compound are performed in vivo.

16. The method of claim 1, wherein the administration of said T-cell activating composition and the administration of said compound are performed separately.

17. The method of claim 1, wherein the administration of said T-cell activating composition and the administration of said compound are performed together.

18. A method for activating T-cells, comprising:
administering to a population of T-cells and monocytes about 0.05 to about 10 mg per day of a composition that inhibits the production or release of intercellular reactive oxygen metabolites (ROM) which is selected from the group consisting of histamine, histamine dihydrochloride, serotonin, dimaprit, clonidine, tolazoline, impornadine, 4-methylhistamine, betazole, and a histamine congener; and
whereby the administration of said composition results in the proliferation of T-cells.

19. The method of claim 18, wherein the composition effective to inhibit the production or release of intercellular reactive oxygen metabolites is selected from the group consisting of histamine, histamine dihydrochloride, serotonin, dimaprit, clonidine, tolazoline, impromadine, 4-methylhistamine, betazole, and a histamine congener.

20. The method of claim 18, wherein the composition is administered at a dose of about 0.1 to about 8 mg/day.

21. A method for activating T-cell proliferation in the presence of monocytes, comprising:
administering to a population of T-cells in the presence of monocytes an effective amount of a first composition selected from the group consisting of a T-cell proliferation enhancing vaccine adjuvant, a T-cell proliferation enhancing vaccine, a T-cell proliferation enhancing peptide, a T-cell proliferation enhancing cytokine selected from the group consisting of IL-1, IL-12, IL-15, IFN-β, and IFN-γ, and a T-cell proliferation enhancing flavonoid; and
administering about 0.05 to about 10 mg per day of a second composition containing at least one compound having H₂-receptor agonist activity which is selected from the group consisting of histamine, histamine dihydrochloride, serotonin, dimaprit, clonidine, tolazoline, impormadine, 4-methylhistamine, betazole, and a histamine congener.

22. The method of claim 21, wherein said second composition comprises histamine.

23. The method of claim 21, wherein said first and second compositions are administered separately.

24. The method of claim 21, wherein the first and second compositions are administered together.

25. The method of claim 21, wherein said administrations are performed in vivo.

26. The method of claim 21, wherein said second composition comprises histamine dihydrochloride.
